(19) United States
(12) Patent Application Publication
(10) Pub. No.: US 2024/0285207 A1
(43) Pub. Date: Aug. 29, 2024

(54) APPARATUS AND METHOD FOR ASSESSING ACTIVE-ESCAPE BIAS IN MAMMALS

(71) Applicant: Centre for Addiction and Mental Health, Toronto (CA)

(72) Inventors: Povilas Karvelis, Toronto (CA); Andreea Diaconescu, Vaughan (CA)

(21) Appl. No.: 18/655,054

(22) Filed: May 3, 2024

Related U.S. Application Data
(63) Continuation of application No. PCT/CA2022/051627, filed on Nov. 3, 2022.
(60) Provisional application No. 63/276,349, filed on Nov. 5, 2021.

(51) Int. Cl. A61B 5/16; A61B 5/00

(52) U.S. Cl. CPC A61B 5/167; A61B 5/7267

(57) ABSTRACT

A series of cues are provided to mammalian subject in association with a predetermined pattern of response states. Responsive to each cue, a physical signal of actuation, or non-actuation within a predetermined time from initiation of the cue, is received and recorded in association with the respective response state. Each response state is an active-escape state, a passive-escape state, an active-avoid state, or a passive-avoid state. The predetermined pattern includes a plurality of sequences and at least one reversal. The physical signals are transformed according to a predefined model incorporating the predetermined pattern to obtain at least one learning variable of the mammalian subject that includes at least one of a belief decay rate and a learning rate, and the predefined model is applied to the learning variable(s) to classify an expected cause of an individual bias of the mammalian subject toward or away from active-escape behaviour.

### CROSS-REFERENCE TO RELATED APPLICATION

This application is a continuation application of PCT Application No. PCT/CA2022/051627, filed on Nov. 3, 2022, entitled APPARATUS AND METHOD FOR ASSESSING ACTIVE-ESCAPE BIAS IN MAMMALS, which claims priority to U.S.

Provisional Application No. 63/276,349 filed on Nov. 5, 2021, the entireties of which are hereby incorporated by reference.

### TECHNICAL FIELD

The present disclosure relates to assessing mammalian behavioral characteristics, and more particularly to assessing active-escape bias in mammals.

### BACKGROUND

Within the active inference framework, Pavlovian and instrumental modes of behavior can be derived from the same central computational goal, which could be thought of as maximizing model evidence, resisting entropy or maintaining homeostasis (Pezzulo et al., 2015). Being nested hierarchically—from reflexive to Pavlovian, to habitual, to instrumental behaviors—different modes of behavior allow a mammal to successfully navigate increasingly more complex environments, but also require more computational and metabolic resources. This poses a problem of bounded rationality (i.e. finding a balance between behavioral accuracy and metabolic costs), which can be resolved by performing Bayesian model averaging (BMA) over the different modes of behavior (FitzGerald et al., 2014). This means that actions are informed by all modes of behavior, whereby the modes with the highest model evidence have the most influence. In these computational terms, a stronger active-escape bias can be understood as resulting from a reduced model evidence for instrumental relative to Pavlovian control.

In active inference, the model evidence of different policies (e.g., Pavlovian vs. instrumental) depends on how well they fulfil outcome priors, which encode the desired outcomes (Friston et al., 2016). Thus, saying that instrumental control has a reduced model evidence is the same as saying that instrumental beliefs have a reduced probability of fulfilling outcome priors—i.e., beliefs are more ‘negative’ in a non-mathematical sense.

### SUMMARY

In one aspect, a method is provided for predicting active-escape bias in a mammalian subject. A series of cues is provided to the mammalian subject. In association with the cues, a physical stimulator adapted to selectively apply an aversive physical stimulus is used to administer to the mammalian subject, according to a predetermined pattern, a series of response states. Each of the response states is associated with a particular one of the cues. Responsive to each of the cues, a physical signal is received from the mammalian subject, via a physical actuator. The physical signal is either actuation of the physical actuator, or non-actuation of the physical actuator within a predetermined time from initiation of the cue. Each received physical signal is recorded in association with the respective response state. Each response state in the series of response states is selected from the group consisting of an active-escape state, a passive-escape state, an active-avoid state and a passive-avoid state. In the active-escape state, the aversive physical stimulus is initially applied, the actuation of the physical actuator will, according to a first probabilistic function, either decrease the duration of the aversive physical stimulus, relative to the non-actuation of the physical actuator, or increase the duration of the aversive physical stimulus, relative to the non-actuation of the physical actuator, and according to the first probabilistic function, the actuation of the physical actuator is more likely to decrease the duration of the aversive physical stimulus relative to the non-actuation of the physical actuator than to increase the duration of the aversive physical stimulus relative to the non-actuation of the physical actuator. In the passive-escape state, the aversive physical stimulus is initially applied, and the actuation of the physical actuator will, according to a second probabilistic function, either decrease the duration of the aversive physical stimulus, relative to the non-actuation of the physical actuator, or increase the duration of the aversive physical stimulus, relative to the non-actuation of the physical actuator, and according to the second probabilistic function, the actuation of the physical actuator is more likely to increase the duration of the aversive physical stimulus relative to the non-actuation of the physical actuator than to decrease the duration of the aversive physical stimulus relative to the non-actuation of the physical actuator. In the active-avoid state, the aversive physical stimulus is initially withheld, and the actuation of the physical actuator will, according to a third probabilistic function, either maintain withholding of the aversive physical stimulus, or initiate application of the aversive physical stimulus, and according to the third probabilistic function, the actuation of the physical actuator is more likely to maintain withholding of the aversive physical stimulus than to initiate application of the aversive physical stimulus. In the passive-avoid state, the aversive physical stimulus is initially withheld, and the actuation of the physical actuator will, according to a fourth probabilistic function, either maintain withholding of the aversive physical stimulus, or initiate application of the aversive physical stimulus, and according to the fourth probabilistic function, actuation of the physical actuator is more likely to initiate application of the aversive physical stimulus than to maintain withholding of the aversive physical stimulus. The predetermined pattern includes at least one first sequence in which the active-escape state is more likely than the passive-escape state and the active-avoid state is more likely than the passive-avoid state, at least one second sequence in which the passive-escape state is more likely than the active-escape state and the passive-avoid state is more likely than the active-avoid state, and at least one reversal between respective ones of the at least one first sequence and the at least one second sequence. The physical signals are transformed according to a predefined model that incorporates the predetermined pattern to obtain at least one learning variable of the mammalian subject, and the predefined model is applied to the learning variable(s) to classify an expected cause of an individual bias of the mammalian subject toward or away from active-escape behavior. The method is characterized in that the learning variable(s) include at least one of a belief decay rate of the mammalian subject and a learning rate of the mammalian subject.

In some embodiments, the predefined model is a structured Bayesian model.

The learning variable(s) may include a stress sensitivity parameter for the mammalian subject and/or a controllability threshold parameter for the mammalian subject.

In some embodiments, the mammalian subject may be a primate, and in particular embodiments the mammalian subject may be a human.

In some embodiments, the classification of the expected cause of the individual bias of the mammalian subject toward or away from active-escape behaviour is presented as a standardized score.

In some embodiments, during the first sequence(s), a likelihood of the active-escape state relative to the passive-escape state varies and a likelihood of the active-avoid state relative to the passive-avoid state varies, and during the second sequence(s), a likelihood of the passive-escape state relative to the active-escape state varies and a likelihood of the passive-avoid state relative to the active-avoid state varies.

In some embodiments, the aversive physical stimulus is selected from the group consisting of aversive aural stimulus, aversive haptic stimulus and aversive olfactory stimulus.

In some embodiments, the physical cue device is a visual cue device. The visual cue device may comprise at least one indicator light, or may comprise at least one display screen. In some embodiments, the physical cue device is an audio cue device.

In some embodiments, the physical cue device is a haptic cue device.

In some embodiments, the physical stimulator is an audio stimulator.

In some embodiments, the physical stimulator is a device that can emit an unpleasant odor.

In some embodiments, the physical stimulator is a device that can apply an unpleasant haptic sensation.

In some embodiments, the physical actuator is one of a button, a lever, a joystick, a switch, a foot pedal, or a touch screen.

In some embodiments, the physical cue device and the physical stimulator comprise a single device.

In other aspects, the present disclosure is directed to an apparatus and to a computer program product for implementing the above-described method.

### BRIEF DESCRIPTION OF THE DRAWINGS

These and other features will become more apparent from the following description in which reference is made to the appended drawings wherein:

FIG. 1 shows an illustrative apparatus for administering a method for predicting active-escape bias in a mammalian subject;

FIG. 2 is a flow chart depicting an illustrative method for predicting active-escape bias in a mammalian subject;

FIG. 3 schematically depicts a computational cycle of active inference and potential perturbations at different stages in the cycle;

FIG. 4 shows a hypothesized brain network;

FIG. 4A shows the hypothesized brain network of FIG. 4, with the proposed computations, possible neural correlates and parameters of interest of a cognitive component of a model for STB;

FIG. 5 schematically depicts an illustrative Avoid/Escape Go/No-go task;

FIG. 6 shows the main parameters of a task component of a model for STB;

FIG. 7A shows trajectories of beliefs and policies in model simulations for a healthy control subject;

FIG. 7B shows trajectories of beliefs and policies under different parameter manipulations in model simulations for increased active-escape biases and other behavioral and cognitive aspects associated with STB;

FIG. 8 shows relevant task performance statistics for various parameter configurations in a model for STB;

FIG. 9 shows dynamics of belief updating and policy probabilities in a model for STB;

FIG. 10 shows an illustrative computer system which may be used as part of the apparatus of FIG. 1 to implement aspects of the method of FIG. 2; and

FIG. 11 shows an illustrative smartphone which may be used as, or as part of, the apparatus of FIG. 1 to implement aspects of the method of FIG. 2.

### DETAILED DESCRIPTION

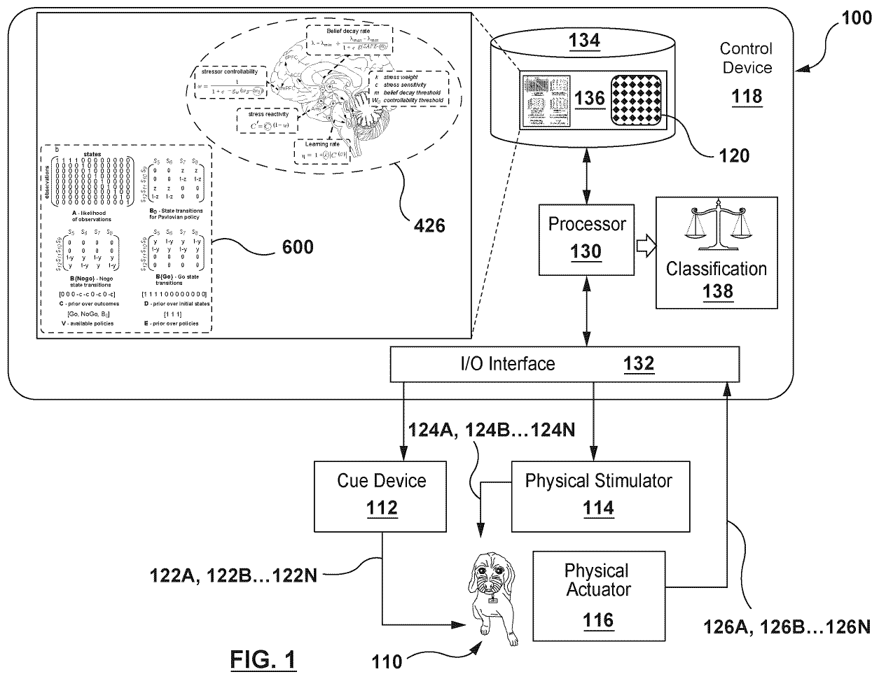

An assessment of a mammalian subject's active-escape bias can provide information useful in managing the mammalian subject, and in some cases can assist in diagnosing medical conditions affecting the mammalian subject. Reference is now made to FIG. 1, which shows an illustrative apparatus, denoted generally by reference 100, for administering a method for predicting active-escape bias in a mammalian subject 110.

The apparatus 100 comprises a physical cue device 112, a physical stimulator 114 adapted to apply an aversive physical stimulus to the mammalian subject 110, and a physical actuator 116, all coupled to a control device 118. The cue device 112 may be a visual cue device, for example, an indicator light or a display screen, or an audio cue device, such as a speaker, or a haptic cue device. The physical stimulator 114 may be, for example, a speaker that can emit an unpleasant noise (aversive aural stimulus), or a device that can emit an unpleasant odor (aversive olfactory stimulus), or a device that can apply an unpleasant haptic sensation (aversive haptic stimulus). In some cases, a single device may function as both the cue device and the physical stimulator (e.g. a speaker). The physical actuator 116 may be, for example, a button, a lever, a joystick, a switch, a foot pedal, or a touch screen, among others.

The control device 118 is configured to use the cue device 112 to provide cues 122A . . . 122N to the mammalian subject 110 and, in association with each cue 122A . . . 122N, use the physical stimulator 114 to administer, according to a predetermined pattern 120, a series of response states 124A . . . 124N to the mammalian subject 110. The response states 124A . . . 124N will be described further below. The control device 118 is further configured to receive from the mammalian subject 110, via the physical actuator 116, physical signals 126A . . . 126N in response to the respective cue 122A . . . 122N. Each of the physical signals 126A . . . 126N is either actuation of the physical actuator 116 (e.g. a “Go” signal) or non-actuation of the physical actuator 116 within a predetermined time from initiation of the respective cue 122A . . . 122N (e.g. a “No-Go” signal). Additionally, the control device 118 is configured to record each received physical signal 126A . . . 126N in association with the respective response state 124A . . . 124N.

The control device 118 may be, for example, a suitably programmed general purpose computer, including any of a desktop computer, laptop computer, tablet computer, or smartphone. For example, the cue device 112 may be a screen, the physical stimulator 114 may be a speaker, and the physical actuator 116 may be a touch screen or button. The control device 118 may also be purpose-built. In one embodiment, the control device comprises at least one processor 130 coupled to an I/O interface 132 and at least one storage 134. The I/O interface 132 manages communication between the processor 130 and the cue device 112, physical stimulator 114 and physical actuator 116, and the storage 134 stores the pattern 120 for the series of response states 124A . . . 124N.

Each response state in the series of response states 124A . . . 124N is selected from the group consisting of an active-escape state, a passive-escape state, an active-avoid state and a passive-avoid state. In both the active-escape state and the passive-escape state, the aversive physical stimulus is initially applied. In both the active-avoid state and the passive-avoid state, the aversive physical stimulus is initially withheld.

In the active-escape state, actuation of the physical actuator will, according to a first probabilistic function, either decrease the duration of the aversive physical stimulus, relative to the non-actuation of the physical actuator, or increase the duration of the aversive physical stimulus, relative to the non-actuation of the physical actuator. In the active-escape state, according to the first probabilistic function, the actuation of the physical actuator is more likely to decrease the duration of the aversive physical stimulus relative to the non-actuation of the physical actuator than to increase the duration of the aversive physical stimulus relative to the non-actuation of the physical actuator. Actuation of the physical actuator is more likely to decrease the duration of the aversive physical stimulus by, for example, substantially immediately terminating the aversive physical stimulus upon actuation of the physical actuator.

In the passive-escape state, actuation of the physical actuator will, according to a second probabilistic function, either decrease the duration of the aversive physical stimulus, relative to the non-actuation of the physical actuator or increase the duration of the aversive physical stimulus, relative to the non-actuation of the physical actuator. In the passive-escape state, according to the second probabilistic function, the actuation of the physical actuator is more likely to increase the duration of the aversive physical stimulus relative to the non-actuation of the physical actuator than to decrease the duration of the aversive physical stimulus relative to the non-actuation of the physical actuator.

In the active-avoid state, actuation of the physical actuator will, according to a third probabilistic function, either maintain withholding of the aversive physical stimulus or initiate application of the aversive physical stimulus. In the active-avoid state, according to the third probabilistic function, the actuation of the physical actuator is more likely to maintain withholding of the aversive physical stimulus than to initiate application of the aversive physical stimulus.

In the passive-avoid state, actuation of the physical actuator will, according to a fourth probabilistic function, either maintain withholding of the aversive physical stimulus or initiate application of the aversive physical stimulus. In the passive-avoid state, according to the fourth probabilistic function, actuation of the physical actuator is more likely to initiate application of the aversive physical stimulus than to maintain withholding of the aversive physical stimulus.

The pattern 120 includes at least one first sequence in which the active-escape state is more likely than the passive-escape state and the active-avoid state is more likely than the passive-avoid state, at least one second sequence in which the passive-escape state is more likely than the active-escape state and the passive-avoid state is more likely than the active-avoid state, and at least one reversal between respective ones of the at least one first sequence and the at least one second sequence. The terms “first” and “second”, as used in this context, distinguish between the two sequences in the pattern 120 and do not imply a particular order; the second sequence may appear before the first sequence, or vice versa. Further, there may be a continuous chain of first sequences and second sequences, each linked by a reversal.

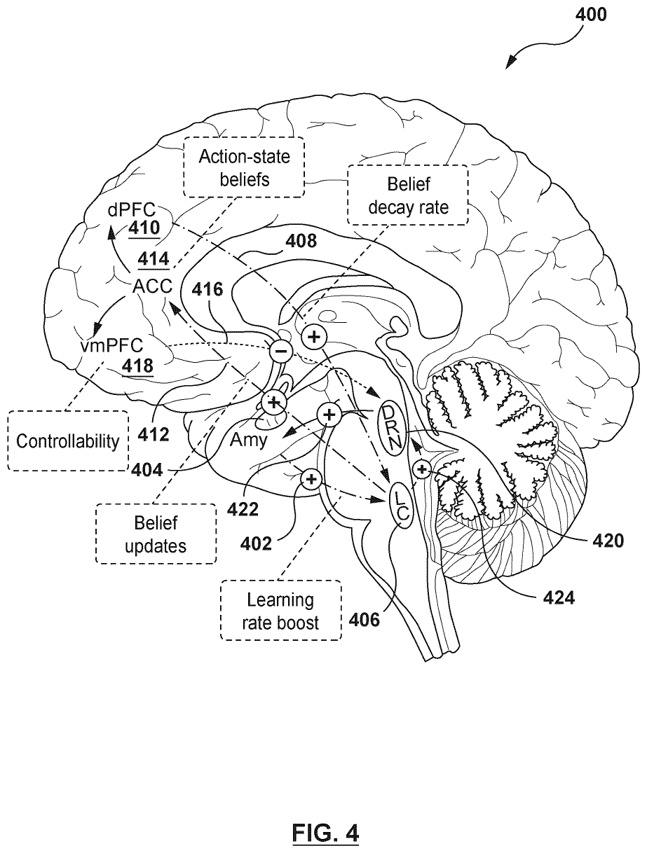

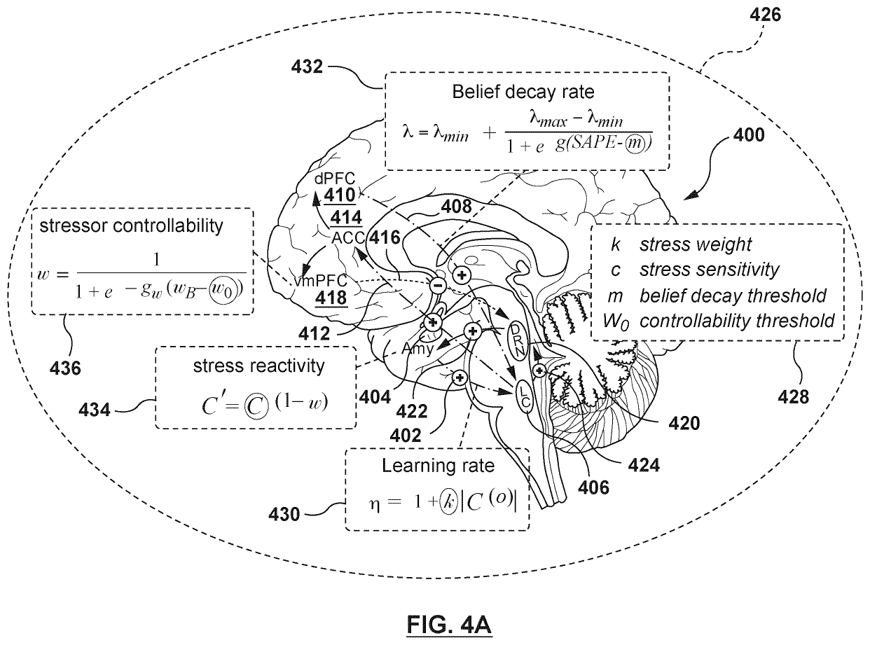

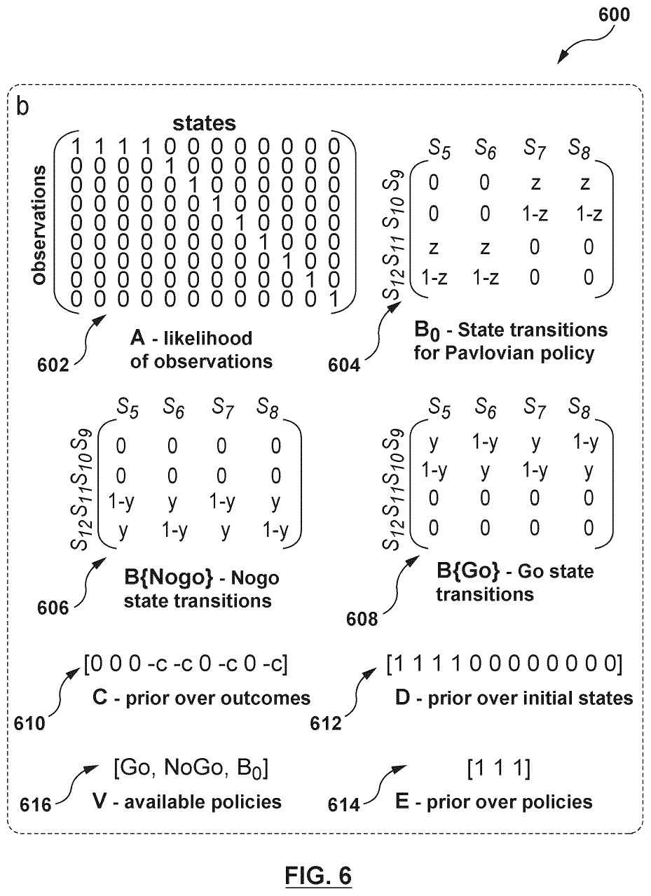

In a preferred embodiment, the control device 118 is further configured to transform the physical signals 126A, 126B . . . 126N according to a predefined model 136 to obtain a classification 138 of an expected cause of an individual bias of the mammalian subject 110 toward or away from active-escape behaviour. As described further below, the predefined model 136 includes a task component 600 (see FIG. 6) and a cognitive component 426 (see FIG. 4A). The predefined model 136 incorporates the predetermined pattern 120 (the first sequence(s), the reversal(s) and the second sequence(s)) and is used to obtain at least one learning variable 428, 430, 432 (see FIG. 4A) of the mammalian subject 110 based on the received physical signal 126A, 126B . . . 126N. The predefined model 136 is then applied to the learning variable(s) 428, 430, 432 to classify an expected cause of an individual bias of the mammalian subject 110 toward or away from active-escape behaviour. The predefined model 136 is characterized in that the learning variable(s) will include either a belief decay rate of the mammalian subject 110 or a learning rate of the mammalian subject 110, or both. The learning variable(s) may further include a stress sensitivity parameter c for the mammalian subject 110, a controllability threshold parameter w₀ for the mammalian subject 110, or both. The belief decay rate, learning rate, stress sensitivity parameter and controllability threshold are discussed further below. In certain preferred embodiments, the predefined model 136 is a structured Bayesian model.

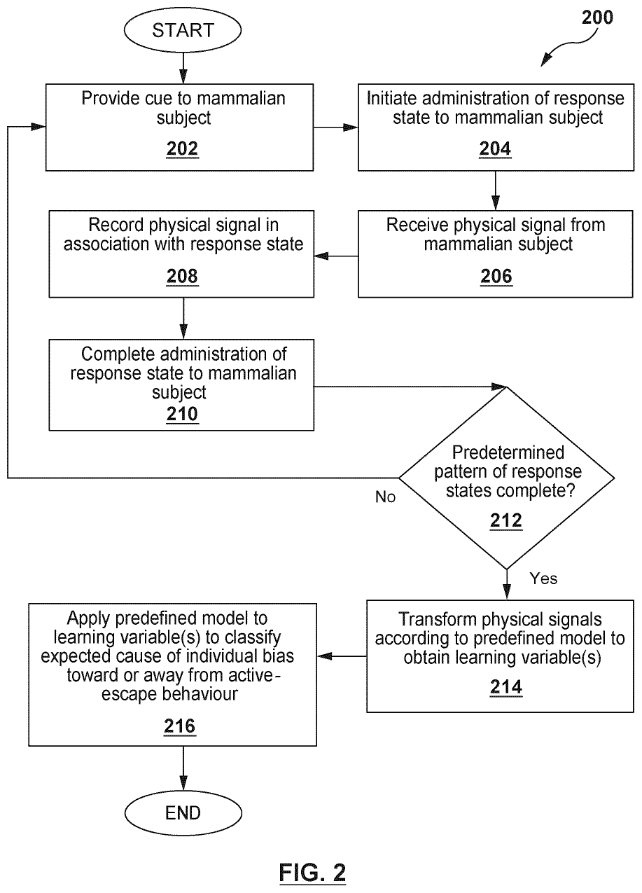

Reference is now made to FIG. 2, in which a method 200 for predicting active-escape bias in a mammalian subject is depicted in flow chart form. The method 200 may be implemented, for example, using the apparatus 100 shown in FIG. 1.

At step 202, the method 200 provides a cue to the mammalian subject (e.g. using cue device 112 in FIG. 1), and at step 204, in association with the cue, the method 200 uses a physical stimulator (e.g. physical stimulator 114 in FIG. 1) adapted to selectively apply an aversive physical stimulus to initiate the administration of a response state to the mammalian subject. The response state whose administration is initiated at step 204 is one of an active-escape state, a passive-escape state, an active-avoid state and a passive-avoid state, and is administered according to a predetermined pattern (e.g. pattern 120 in FIG. 1). Steps 202 and 204 are shown sequentially, but may be performed substantially simultaneously.

An active-escape state is one in which the aversive physical stimulus is initially applied and actuation of a physical actuator will, according to a first probabilistic function, either increase or decrease the duration of the aversive physical stimulus, relative to the non-actuation of the physical actuator. For the active-escape state, the first probabilistic function provides that actuation of the physical actuator is more likely to decrease than to increase the duration of the aversive physical stimulus relative to non-actuation of the physical actuator.

A passive-escape state is one where the aversive physical stimulus is initially applied and actuation of the physical actuator will, according to a second probabilistic function, either decrease or increase the duration of the aversive physical stimulus, relative to the non-actuation of the physical actuator. Within the passive-escape state, the second probabilistic function provides that the actuation of the physical actuator is more likely to increase than to decrease the duration of the aversive physical stimulus relative to non-actuation of the physical actuator.

An active-avoid state is one in which the aversive physical stimulus is initially withheld and actuation of the physical actuator will, according to a third probabilistic function, either maintain withholding of the aversive physical stimulus or initiate application of the aversive physical stimulus. For the active-avoid state, the third probabilistic function provides that actuation of the physical actuator is more likely to maintain withholding of the aversive physical stimulus than to initiate application of the aversive physical stimulus.

A passive-avoid state is one where the aversive physical stimulus is initially withheld and actuation of the physical actuator will, according to a fourth probabilistic function, either maintain withholding of the aversive physical stimulus or initiate application of the aversive physical stimulus. Within the fourth probabilistic function, actuation of the physical actuator is more likely to initiate application of the aversive physical stimulus than to maintain withholding of the aversive physical stimulus.

At step 206, the method 200 receives from the mammalian subject, responsive to the cue, a physical signal. The physical signal received at step 206 is either actuation of a physical actuator (e.g. physical actuator 116 in FIG. 1), or non-actuation of the physical actuator within a predetermined time from initiation of the cue (e.g. a “Go” or “No-Go” signal). At step 208, the method 200 records the physical signal received at step 206 in association with the respective response state initiated at step 204 (e.g. by way of I/O interface 132, processor 130 and data store 134 in FIG. 1).

At step 210, the method 200 completes the administration of the response state initiated at step 204 by applying the respective probabilistic function to the physical signal received at step 206. Where the response state whose administration was initiated at step 204 is an active-escape state, step 210 applies the first probabilistic function to either increase or decrease the duration of the aversive physical stimulus, relative to the non-actuation of the physical actuator. Where the response state whose administration was initiated at step 204 is a passive-escape state, step 210 applies the second probabilistic function to either decrease or increase the duration of the aversive physical stimulus, relative to the non-actuation of the physical actuator. Where the response state whose administration was initiated at step 204 is an active-avoid state, step 210 applies the third probabilistic function to either maintain withholding of the aversive physical stimulus or initiate application of the aversive physical stimulus. Where the response state whose administration was initiated at step 204 is a passive-avoid state, step 210 applies the fourth probabilistic function to either maintain withholding of the aversive physical stimulus or initiate application of the aversive physical stimulus.

Although step 208 is shown as preceding step 210, step 208 may alternatively be carried out after step 210, or substantially simultaneously therewith.

At step 212, the method 200 checks whether the predetermined pattern (e.g. pattern 120 in FIG. 1) of response states has been completed. As noted above, the predetermined pattern includes at least one first sequence in which the active-escape state is more likely than the passive-escape state and the active-avoid state is more likely than the passive-avoid state, at least one second sequence in which the passive-escape state is more likely than the active-escape state and the passive-avoid state is more likely than the active-avoid state and at least one reversal between respective ones of the first sequence(s) and the second sequence(s). If there is more than one first sequence and/or second sequence, there may be a plurality of reversals. Optionally, during the first sequence(s) the likelihood of the active-escape state relative to the passive-escape state varies and the likelihood of the active-avoid state relative to the passive-avoid state varies, and during the second sequence(s) the likelihood of the passive-escape state relative to the active-escape state varies and the likelihood of the passive-avoid state relative to the active-avoid state varies.

If the predetermined pattern has not yet been completed (“no” at step 212), the method 200 returns to step 202 to provide another cue and then proceeds to step 204 to initiate the administration of the next response state in the predetermined pattern. Once the predetermined pattern is completed (“yes” at step 212), the method 200 proceeds to step 214. Thus, over multiple iterations of steps 202 through 210, the method 200, in association with the cues (step 202), uses the physical stimulator to administer to the mammalian subject, according to a predetermined pattern, a series of response states (steps 204 and 210) each associated with a particular one of the cues and, responsive to each of the cues, receives, from the mammalian subject, via the physical actuator, a physical signal (step 206) and records each received physical signal in association with the respective response state (step 208).

At step 214, after the predetermined pattern is completed (“yes” at step 212), the method 200 transforms the physical signals according to a predefined model (e.g. model 136) in FIG. 1) to obtain at least one learning variable of the mammalian subject. As discussed above, the predefined model incorporates the predetermined pattern 120 (the first sequence(s), the reversal(s) and the second sequence(s)). At step 216, the method applies the predefined model to the learning variable(s) to classify an expected cause of an individual bias of the mammalian subject toward or away from active-escape behaviour. Classification (e.g. classification 138) of the expected cause of the bias of the mammalian subject toward or away from active-escape behaviour may be presented as a standardized score. The learning variable will preferably include one, or both, of a belief decay rate of the mammalian subject and a learning rate of the mammalian subject, and may further include one or both of a stress sensitivity parameter for the mammalian subject and a controllability threshold parameter for the mammalian subject. The belief decay rate, learning rate, stress sensitivity parameter and controllability threshold parameter are discussed further below. The predefined model may be a structured Bayesian model, and nested probabilities may be incorporated into the model. Of note, the information processing system that transforms the physical signals according to the predefined model may be part of the apparatus 100, or may be a different system which receives the physical signals.

In the illustrative embodiment, the mammalian subject 110 in FIG. 1 is depicted (with a respectful nod to Ivan Pavlov) as a dog named “Coffee” to illustrate that the method 200 may be applied in respect of any mammal that can be trained to use a suitable physical actuator 116 in response to a cue 122A, 122B . . . 122N. The method 200 has particular application in respect of primates, and even more particular application in respect of humans (humans being a particular instance of primate). In such cases, classification of the bias of the human subject toward or away from active-escape behaviour may be presented as a standardized score to assist a clinician in diagnosing or treating a human patient. Thus, the apparatus 100 may be considered a form of diagnostic instrument.

While not limited to such applications, a potential application of the method 200 shown in FIG. 2 for humans is to assist in diagnosis or prediction of potential suicidal thoughts. While an illustrative theoretical framework for applying the method 200 in this context is described, the method 200 and its application should not be construed as being limited to such applications, and may be used more generally to classify an expected cause of an individual bias of a mammalian subject toward or away from active-escape behaviour. Moreover, while certain references are cited to facilitate understanding of this illustrative theoretical framework, citation of any reference anywhere in this document is not an admission that such reference is citable as prior art under any relevant legal framework. Further, citation of any reference within this document is not an admission that such reference is relevant to assessing novelty or inventiveness of the claims, even if such reference is legally citable as prior art.

Suicide is the second leading cause of death among young adults and among the top ten causes of death across all ages worldwide (Naghavi et al., 2017). Despite decades of research seeking to identify risk factors of suicidal thoughts and behaviors (STB), their predictive ability remains limited (Large et al., 2016; Franklin et al., 2017). Some of the main risk factors include the following: prior psychiatric diagnosis, treatment history, family history of psychopathology, prior self-injurious thoughts and behaviors, substance use and psychosocial stress. However, multivariate suicide risk models based on these factors do not have sufficient sensitivity and specificity in predicting suicide and, even more importantly, lack mechanistic insight to offer clinically useful guidance on selecting optimal individualized interventions (Kessler et al., 2020). As a result, current clinical practice is in need of objective and reliable measures of suicide risk to not have to rely on self-reports, with ˜50% of adults not disclosing their suicidal thoughts and remaining invisible for suicide prevention efforts (Mérelle et al., 2018).

In recent years, cognitive theories have proposed several explanations for the progression from emotional distress to suicidal ideation, and to suicide attempts (Van Orden et al., 2010; Klonsky and May 2015; O'Connor and Kirtley, 2018; Bryan et al., 2020). At the core of these proposals is the recognition that suicide can be viewed as a means to escape mental pain (psychache) (Baumeister, 1990; Verrocchio et al., 2016). While mental pain and hopelessness contribute to suicidal ideation, other factors, collectively termed ‘acquired capability for suicide’ (e.g., increased physical pain tolerance, access to lethal means), mediate the transition from ideation to suicide attempt (Klonsky et al., 2018). While providing useful high-level insights into the different psychological and environmental factors associated with suicidality, the verbal nature of these theories limits their predictive power (Millner et al., 2020; Meehl, 1990). Natural language is inherently vague resulting in intercorrelated constructs on which the theories rest, making it difficult to corroborate or refute them (Millner et al., 2020; Meehl, 1990). This calls for formal theories of suicidality which can be expressed computationally and which can define these constructs operationally (Millner et al., 2020; Dombrovski and Hallquist, 2021). Computational models could allow for a quantification of suicide risk and offer a more mechanistic insight for developing personalized clinical interventions (Nair et al., 2020; 27 Millner et al., 2020) and could also help bridge different levels of analysis and establish mechanistic links between behavioral, cognitive, neural and even genetic variables, offering a more integrated understanding of the factors underlying vulnerability to STB (Huys et al., 2021).

One principled way of building such models is to investigate vulnerability to STB through the lens of normative theories of learning and decision making in computational neuroscience (Dombrovski and Hallquist, 2021, 2017). Collectively, STB has been associated with deficits in cognitive control (Richard-Devantoy et al., 2014) and impaired probabilistic learning in the context of rewards and punishments, including impaired delay discounting (Bridge et al., 2015), impaired reversal learning (Dombrovski et al., 2010) and impaired value comparison during the choice process (Dombrovski et al., 2019). Outside of the laboratory, this is corroborated by findings of heightened suicide risk in gambling disorders (Karlsson and Hakansson, 2018; Jolly et al., 2021). Behavioral insensitivity to adverse consequences and heightened sensitivity to internal emotional states have also been linked to suicide attempts (Szanto et al., 2014). Together, these findings have led to a proposal of increased Pavlovian over instrumental control as being an important contributing factor to vulnerability to STB (Dombrovski and Hallquist, 2017, 2021). The Pavlovian controller rigidly specifies stimulus-response mappings regardless of outcomes, such as actively escaping proximal threats and avoiding distal threats, resulting in a rather reflexive behavior. In contrast, the instrumental control specifies stimulus-action-outcome mappings enabling one to adapt behaviors to environmental contingencies and maximize desired outcomes, which can be thought of as goal-directed behavior. In line with the idea of increased Pavlovian biases, a recent study by Millner et al. (2019) found STB to be associated with an increased active-escape bias in an Avoid/Escape Go/No-go task with aversive sound stimuli. In this study, the STB group was more biased towards choosing an active (Go) response in the presence of an aversive sound (in Escape condition), even when withholding the response (in No-Go condition) was the correct response.

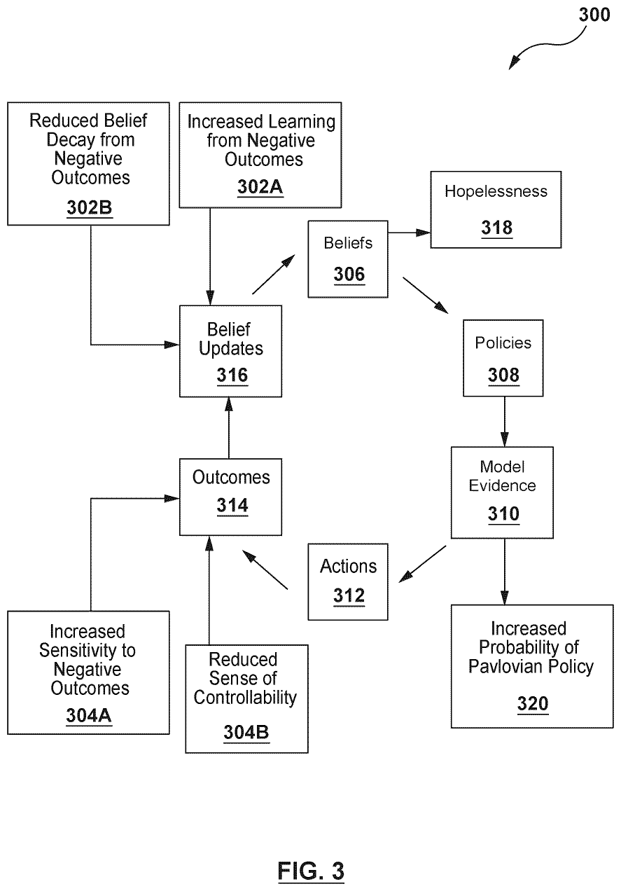

The present disclosure describes an application of the method 200 described above in the context of FIG. 2 to implement an assessment protocol based on a proposed computational mechanism for how the increased Pavlovian biases in STB could result from impaired probabilistic learning, as shown in FIG. 3.

FIG. 3 depicts a computational cycle 300 of active inference (306, 308, 310, 312, 314, 316) and potential perturbations 302A, 302B, 304A, 304B at different stages in the cycle 300. The cycle 300 of active inference includes beliefs 306 about state transitions under different policies, and policies 308 that fulfill outcome priors get higher model evidence. Model evidence 310 of Pavlovian vs. instrumental policies determine their probabilities, and chosen actions 312 are proportional to policy probabilities. The outcomes 314 lead to belief updates 316 for the beliefs 306. The perturbations include increased learning from negative outcomes 302A and reduced belief decay (unlearning) in response to unexpected outcomes 302B, which affect belief updates 316. The perturbations further include increased sensitivity to negative outcomes 304A and reduced sense of controllability 304B, which affect the impact of the outcomes 314. These perturbations 302A, 302B, 304A, 304B can give rise to hopelessness 318-a belief that any taken action will lead to undesired states—and an increased influence of Pavlovian relative to instrumental modes of behavior 320, both of which are associated with suicidality.

Without being limited by theory, it is believed that impaired probabilistic learning is mediated by hopelessness (a belief that there is nothing one can do to make things better), which is one of the most robust factors of suicide risk (Isometsä, 2014; May et al., 2020). To this end, active inference is applied as the most general neurocomputationally-principled framework that integrates perception, action and learning into a continuous loop of information processing (Friston et al., 2013). The principle guiding this information processing is maximization of (Bayesian) model evidence for one's model of the world, which simultaneously reduces uncertainty about the world and achieves desired outcomes. By operationalizing hopelessness as predominantly negative instrumental beliefs (i.e., with all available actions believed to have low probability of leading to the desired states), an increased Pavlovian control emerges as a straightforward consequence of the drive to maximize model evidence. Again without being limited by theory, it is proposed that four different perturbations within the context of aversive learning could give rise to hopelessness itself: (1) an increased learning from aversive outcomes, (2) a reduced belief decay in response to unexpected outcomes, (3) an increased stress sensitivity parameter c and (4) a reduced sense of stressor controllability (higher controllability threshold w₀).

These proposals stem from the consideration of neurocircuits implicated in STB. Research on suicide neuromarkers point to the circuits underlying stress response, implicating the locus coeruleus-norepinephrine (LC-NE) and the dorsal raphe nucleus-serotonin (DRN-5-HT) systems (Mann and Rizk, 2020; Oquendo et al., 2014; van Heeringen and Mann, 2014). More broadly, neuroimaging findings are converging on fronto-limbic regions involved in emotion regulation and cognitive control, including the amygdala (Amy), the anterior cingulate cortex (ACC), the dorsal prefrontal cortex (dPFC) and the ventromedial prefrontal cortex (vmPFC) among other regions (Schmaal et al., 2020; Balcioglu and Kose, 2018). However, computational models linking these neuromarkers with the behavioral markers are still missing. The proposed computational perturbations in STB could be related to how the LC-NE together with the Amy, the dPFC and the ACC mediate learning in response to acute stress and volatility as well as how the DRN-5-HT together with the vmPFC regulate stress responses based on the perceived controllability of the aversive stimulus.

Reference is now made to FIG. 4, which shows a hypothesized brain network 400 to support the proposed perturbations. Norepinephrine (402, 408, 412) modulates belief updates while serotonin (416, 422) is involved in mediating the effects of stressor controllability. Acute stress leads to increases in the learning rate, which is associated with connectivity 402 between the Amy 404 and the LC 406 (Amy-LC connectivity 402) (Uematsu et al., 2017; Jacobs et al., 2020). Environmental volatility-here assuming state-action prediction errors (SAPEs) as a proxy for environmental change-drives decay of previously learned associations and is mediated by connectivity 408 between the dPFC 410 and the LC 406 (dPFC-LC connectivity) (Sales et al., 2019; Clewett et al., 2014). Projections 412 from the LC 406 to the ACC 414 mediate action-dependent state transition belief updates (Tervo et al., 2014; Sales et al., 2019), which are encoded in the ACC 414 (Akam et al., 2021; Holroyd and Yeung, 2012). Finally, controllability of aversive outcomes, which depends on current beliefs about the state transitions under different actions, reduces aversiveness by inhibiting amygdala (Amy) 404 activation via a connection 416 from the vmPFC 418 to the DRN 420 and a connection 422 from the DRN 420 to the Amy 404 (the vmPFC-DRN-Amy circuit) (Maier and Seligman, 2016; Kerr et al., 2012).

The model was validated by running model simulations in a probabilistic Avoid/Escape Go/No-go task, demonstrating how the proposed perturbations lead to hopelessness, increased Pavlovian control and increased active-escape bias-replicating recent empirical findings by Millner et al. (2019). This serves as a proof of concept and produces a computational hypothesis space which can be investigated experimentally and which might speak to different subtypes of suicidal behaviour: impulsive versus planful attempts (Schmaal et al., 2020; Dombrovski and Hallquist, 2017; Bernanke et al., 2017).

Thus, in one aspect, the present disclosure will operationalize hopelessness, which is one of the most robust suicide risk factors (May et al., 2020; Isometsä, 2014), as strong negative instrumental beliefs about state transitions.

To understand how hopelessness arises, consider the dynamics of belief updating, i.e. learning. Having predominantly negative beliefs (hopelessness) implies either a predominantly aversive environment or preferential learning from aversive events. Asymmetries in how positive and negative outcomes drive learning (i.e. affective bias) have been implicated in mood disorders (Pulcu and Browning, 2017; Clark et al., 2018; Pulcu and Browning, 2019), with negative outcomes having larger effect on learning than positive outcomes (Mathews and MacLeod, 2005; Eshel and Roiser, 2010). Conversely, in the general population learning is driven more strongly by positive outcomes (Sharot and Garrett, 2016). In STB, research on learning from negative vs. positive outcomes is scarce, but a recent study showed STB to be associated with faster processing of negative stimuli (Harfmann et al., 2019).

While the learning rate can be affected by multiple neuromodulatory systems, when it comes to adjusting the learning rate in response to acute stress and volatility, the LC-NE system plays an important, if not central, role (Pulcu and Browning, 2019; Cook et al., 2019; Silvetti et al., 2018; Jepma et al., 2016; Lawson et al., 2020). Previous influential theories of LC 406 function were founded on the assumption that LC-NE cells behave homogeneously (Yu and Dayan, 2005; Bouret and Sara, 2005). However, recent research emphasizes that LC 406 firing properties are not topographically homogeneous and rather that the LC 406 is comprised of largely non-overlapping target-specific subpopulations of neurons (Poe et al., 2020; Chandler et al., 2019). Importantly, aversive learning is mediated by Amy-LC connectivity (Sterpenich et al., 2006; Uematsu et al., 2017; Jacobs et al., 2020), whereas connectivity between the prefrontal cortex (PFC) regions and the LC 406 has been found to represent belief decay or ‘unlearning’, which is necessary for faster adaptation to environmental change or volatility (Uematsu et al., 2017; Sales et al., 2019). Relevant here, dPFC-LC connectivity 408 has been shown to encode learning from unpredictable feedback (Clewett et al., 2014) and response conflict resolution (Köhler et al., 2016; Grueschow et al., 2020). The dorsolateral PFC (dlPFC) itself has been associated with state prediction error (as opposed to reward prediction error) (Gläscher et al., 2010). LC projections 412 to the ACC 414 have been shown to mediate updates of action-dependent beliefs about the environment (Tervo et al., 2014; Sales et al., 2019), with the ACC encoding such beliefs (Akam et al., 2021; Holroyd and Young, 2012). This is consistent with the findings that ACC 414 activity correlates with reward expectation, prediction errors, learning rate and volatility (Rushworth and Behrens, 2008), with these learning variables engaging the ACC 414 primarily in the context of learning about the value of instrumental actions (Matsumoto et al., 2007).

Several lines of evidence suggest the aforementioned networks to be implicated in suicidality (Schmaal et al., 2020; Oquendo et al., 2014). Studies have reported fewer LC 406 neurons, LC 406 overactivity and depletion of NE, all of which are thought to be associated with a dysregulated stress response (Oquendo et al., 2014; van Heeringen and Mann, 2014). The Amy 404 is reported to show increased resting state functional connectivity (Kang et al., 2017) with some structural MRI studies also reporting larger Amy 404 volumes (Monkul et al., 2007; Spoletini et al., 2011). Studies on the dPFC 410 report reduced volumes (Ding et al., 2015), decreased resting regional cerebral blood flow (rCBF) (Willeumier et al., 2011) and reduced activation during error processing (Vanyukov et al., 2016). ACC volumes are also reported to be reduced, with reductions in rostral ACC (rACC) being most significant (Wagner et al., 2011). In a risk aversion task, suicide attempters showed a blunted subgenual ACC 414 activation in response to potential gains (Back et al., 2017), a reduced ACC 414 response to sad faces and an increased response to wins versus loses (Olié et al., 2015). Finally, a recent study found greater rACC-Amy functional connectivity to be associated with suicidal ideation and previous suicide attempts (Alarcón et al., 2019).

Here, without being limited by theory, it is proposed that a disruption in any part of the Amy-dPFC-LC-ACC network (402, 408, 412) could lead to hopelessness, increased Pavlovian and active-escape bias, increasing the risk of STB. Specifically, consider two possible perturbations. First, an increased Amy 404 response to negative outcomes would increase learning from negative outcomes (i.e., negative affective bias), which may lead to more negative beliefs (hopelessness) and thus stronger Pavlovian influences. This is supported by increased learning rate in STB observed in an aversive learning task (Millner et al., 2019). Second, reduced activity in the dPFC 410 in response to state-action prediction errors would result in less belief decay allowing negative experiences to accumulate, thus also resulting in hopelessness and stronger Pavlovian biases. Interestingly, impairments in the dPFC 410 have been mostly associated with planful suicides (Schmaal et al., 2020), which would be in agreement with the cognitive rigidity induced by reduced belief decay that is considered here.

Recent work has shown that controllability of action outcomes governs arbitration between Pavlovian and instrumental control in line with BMA (Dorfman and Gershman, 2019). These effects were found to be associated with frontal midline theta power, which suggests involvement of the mPFC and the ACC 414 (Csifcsák et al., 2020). Furthermore, it has been proposed that dorsal ACC (dACC) could be understood as encoding the expected value of control (Shenhav et al., 2013). This is very similar to what is proposed in relation to hopelessness. Indeed, controllability and hopelessness are very closely related constructs. Uncontrollable aversive stimulation has been used to study learned helplessness, from which the construct of hopelessness has been derived (Liu et al., 2015). Another extensively studied effect of controllability is that of modulating the stress response. Stressor controllability has been associated with the vmPFC-DRN-Amy network 416, 422, and thus with 5-HT-modulated stress response (Maier and Seligman, 2016; Kerr et al., 2012; Hiser and Koenigs, 2018). More specifically, stressor controllability activates the vmPFC 418, which then inhibits DRN 420, which in turn reduces Amy 404 activation in response to a stressor (Maier and Seligman, 2016). Relevant here, recent studies also show this effect to be associated with successful instrumental learning (Collins et al., 2014; Wanke and Schwabe, 2020).

Considering these findings, the present disclosure introduces a computational distinction between hopelessness and controllability. As defined earlier, hopelessness corresponds to negative instrumental state-action beliefs that are encoded in the ACC 414 and are arrived at via LC-mediated updates 412. Controllability, on the other hand, is associated with the vmPFC-DRN-Amy network 416, 422 and thus with 5-HT-modulated stress response. It is assumed that the instrumental state-transition beliefs encoded in the ACC 414 are the main input for estimating controllability in the vmPFC 418, as discussed further below. This provides a computational link between the NE-modulated and the 5-HT-modulated variables and allows hopelessness and controllability to be distinct but coupled. Interestingly, projections 424 from the LC 406 to the DRN 420 have also been shown to regulate 5-HT release (Pudovkina et al., 2003) and be necessary for developing learned helplessness following uncontrollable stressor exposure (Grahn et al., 2002), providing another point of interaction between the two neuromodulatory systems, which is not specifically addressed here.

In suicidality, a large body of research points to deficits in the serotonergic system (van Heeringen and Mann, 2014; Oquendo et al., 2014). While lower 5-hydroxyindoleacetic acid (5-HIAA) levels—a major serotonin metabolite—in the cerebrospinal fluid (CSF) suggest reduced overall serotonergic activity (Mann et al., 2006), serotonin in the brainstem is found to be elevated (Bach et al., 2014), with serotonergic action being elevated in the DRN 420 due to less reuptake (Arango et al., 2001). Furthermore, studies also report elevated serotonin binding in the Amy 404 (Hrdina et al., 1993) and fewer serotonin transporters in the vmPFC 418 and the ACC 414 (Mann et al., 2000). A recent study has also found a history of suicide attempts to be associated with a diminished functional connectivity between vmPFC 418 and Amy 404 (Wang et al., 2020). Together, these findings are consistent with an increased 5-HT-mediated stress response in suicidality.

The present disclosure proposes that a reduced sense of controllability stemming from impairments in the vmPFC-DRN-Amy network (416, 422) can lead to a stronger Amy 404 activation in response to stress, thus increasing learning from negative outcomes and leading to hopelessness and stronger Pavlovian biases. Impairments in the vmPFC 418 have been associated with impulsive suicide attempts (Schmaal et al., 2020), which would be in line with larger belief updates in response to stressors.

The foregoing description lays out a conceptual picture of the proposed model by considering various computational and neurobiological findings. One illustrative computational implementation will now be described by focusing on an Avoid/Escape Go/No-go task, which may be implemented, for example, using the apparatus 100 described above in the context of FIG. 1. Note that the implementation of the model (e.g. model 136) is not at the level of neural dynamics but rather at the higher level of computational mechanisms underwritten by such dynamics (cf. Marr's levels of analysis (Marr and Poggio, 1976)). However, the active inference framework has deep connections to neurobiology and has recently been applied to understanding a whole range of psychiatric conditions (Smith et al., 2021), including the effects of noradrenergic and serotonergic drugs in depression (Constant et al., 2021).

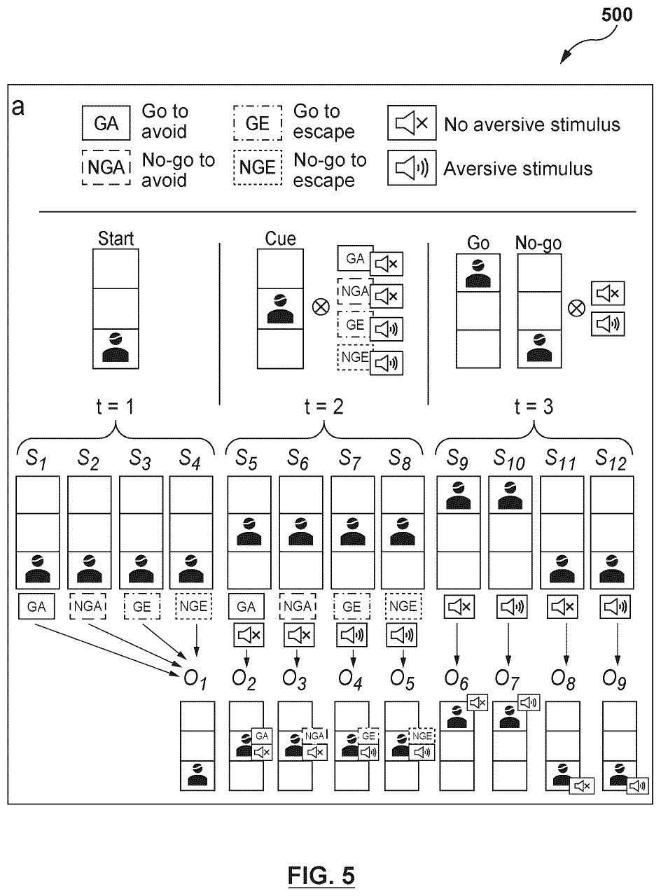

Reference is now made to FIG. 5, which schematically depicts an illustrative Avoid/Escape Go/No-go task 500. The task 500 has four cues corresponding to the 2×2 (Go/No-go×Avoid/Escape) factorial task structure, with 2 possible outcomes: aversive or neutral. For simplicity, an active inference scheme for discrete Markovian models (Friston et al., 2016) is used, such that there are discrete time steps (t), discrete states (s), and discrete actions and observations (o). Each trial is divided into three time steps. At t=1, the agent is in one of four possible hidden states (s₁₋₄) with no observations available (o₁). At t=2, the agent is presented with one of the four cues, which correspond to one of the four conditions resulting from the 2×2 (Go/No-go×Avoid/Escape) factorial design. This corresponds to step 202 of the method 200 shown in FIG. 2. Presentation of the cue is associated with one of four possible hidden states (s₅₋₈) and observations (o₂₋₅). The hidden states are one of active-escape state, a passive-escape state, an active-avoid state and a passive-avoid state, as described above. Thus, these four hidden states correspond to step 204 of the method 200 (initiating administration of the response state). In the active-escape state and the passive-escape state, an aversive stimulus (e.g. an aversive sound) is present throughout the decision phase; the aversive stimulus is absent in the active-avoid state and the passive-avoid state. At t=2, responsive to the cue, the agent chooses what action to take (Go or No-go) which then leads to one of four possible states (s₉₋₁₂) and observations (o₆₋₉). The choice is indicated via a physical actuator (e.g. physical actuator 116), and corresponds to step 206 of the method 200. The choice is recorded, corresponding to step 208. At t=3, the agent observes the final outcome of a trial, either aversive or neutral. This means that in the active-avoid state and the passive-avoid state, a correct action (or inaction) leads to no aversive sound, while in the active-escape state and the passive-escape state, a correct action (or inaction) results in the discontinuation of the aversive sound. This corresponds to step 210 of the method 200.

Reference is now made to FIG. 6, which shows the main parameters of the task component 600 of the model (e.g. model 136). Due to the salience of the aversive stimulus, the task component 600 of the model assumes no uncertainty in the likelihood of observations, A, denoted by reference 602. State transition probabilities from t=2 to t=3 for Pavlovian policy, B₀, denoted by reference 604, were implemented to reflect beliefs that No-Go response in Avoid and Go response in Escape conditions will lead to no aversive stimulus. The strength of this belief is captured by the z parameter. State transition probabilities from t=2 to t=3 for instrumental (Go/No-go) policies B{Nogo} (denoted by reference 606), B{Go} (denoted by reference 608) represent the objective probabilities of state transitions controlled by parameter y. For the simulations presented in this disclosure, y was set to 0.8, meaning that correct response by the agent led to the neutral outcome 80% of the time. This is merely one illustrative implementation, and is not limiting. For subjective beliefs about state transitions y was initialized with 0.5 to correspond to a uniform prior; again, this is illustrative and not limiting. Prior over outcomes (C), denoted by reference 610, assumed that the agent does not like outcomes 4, 5, 7 and 9 (all of which involve the aversive stimulus). The strength of this preference of neutral outcomes is captured by parameter c. Prior over initial states D, denoted by reference 612, was assumed to be uniform. Finally, prior over policies E, denoted by reference 614, was also assumed to be uniform across the available Go, No-go and Pavlovian policies (π). General Matlab code implementing Active Inference can be found at https://www.fil.ion.ucl.ac.uk/spm/software/spm12/which is hereby incorporated by reference. Code from this toolbox (spm mdp VB.m) was modified to perform the simulations presented in this disclosure and can be found at: https://github.com/frank-pk/STB AEGNG AI, which is hereby incorporated by reference.

When choosing an action at t=2, the agent relies on available policies V: instrumental Go/No-go and Pavlovian, as denoted by reference 616. Probabilities of these policies depend on the underlying beliefs about likelihood of observations, A (602), state transitions—B{Go} (608), B{Nogo} (606), B₀ (604)—as well as prior beliefs over outcomes (i.e., preferences), C (610). In other words, probabilities of policies depend on model evidence that each set of beliefs provides, where model evidence is approximated with variational free energy.

Bayesian inference of hidden states and model parameters, x, given sensory observations o:

$$P ( x \vert o ) = \frac{P ( o \vert x ) P ( x )}{P ( o )}$$

The exact posterior P(x|o) is hard to compute, but can be easily approximated with some function Q(x) by minimizing KL-divergence between P(x|o) and Q(x), which can be achieved by minimizing free energy, F:

$$\begin{matrix}F = - \int dxQ ( x ) ln [ \frac{P ( o , x )}{Q ( x )} ] = {E}_{Q ( x )} [ ln Q ( x ) - ln P ( o , x ) ] \\ = {D}_{KL} [ Q ( x ) ∥ P ( x ) ] - {E}_{Q ( x )} [ ln P ( o \vert x ) ]\end{matrix}$$

The agent's generative model can be formulated as Partially Observable Markov Decision Process (POMDP) with joint probability over observations õ and causes of those observations X=(\tilde{s}, π, θ), with θ=(a, b, c, d, e, β) containing the set of all model parameters:

$$P ( \tilde{o} , \tilde{s} , π , θ ) = P ( π ) P ( θ ) {\prod }_{t = 1}^{T} P ( {o}_{t} \vert {s}_{t} ) P ( {s}_{t} \vert {s}_{t - 1} , π )$$

The agent's approximate posterior over hidden states \tilde{s} and parameters takes the form:

$$Q ( \tilde{s} , π , θ ) = P ( π ) P ( θ ) {\prod }_{t = 1}^{T} P ( {s}_{t} \vert π )$$

These can be combined to obtain:

$$\begin{matrix}F = {E}_{Q} [ ln ( Q ( π ) Q ( θ ) {\prod }_{t = 1}^{T} Q ( {s}_{τ} \vert π ) ) - ln ( P ( π ) P ( θ ) {\prod }_{t = 1}^{T} P ( {o}_{t} \vert {s}_{t} ) P ( {s}_{t} \vert {s}_{t - 1} , π ) ) ] \\ = {D}_{KL} [ Q ( π ) ∥ P ( π ) ] + {D}_{KL} [ Q ( θ ) ∥ P ( θ ) ] + \\ {E}_{Q} [ {\sum }_{τ = 1}^{T} ln ( \frac{Q ( {s}_{τ} \vert π )}{P ( {s}_{τ} \vert {s}_{τ - 1} , π )} - ln P ( {o}_{τ} \vert {s}_{τ} ) ) ]\end{matrix}$$

(see Da Costa et al. (2020) for more details).

After each trial, the agent updates their beliefs depending on the outcome in that trial. Since there is no ambiguity about observations due to their saliency, all learning is assumed to concern only state transition probabilities B{Go} (608), B{Nogo} (606), B₀ (604). Columns in the matrices for B{Go} (608), B{Nogo} (606), B₀ (604) are Dirichlet distributions parameterized with concentration parameters b, such that for action u, B(u)=Dir(b(u)). Concentration parameters can be interpreted as the number of times various combinations of state transitions have been observed, which effectively captures both the probability and the confidence in that probability. At the end of each trial, state transition concentration parameters are updated via:

$${b}_{i} ( u ) = {b}_{i - 1} ( u ) + η {\sum }_{τ , p} {π}_{τ - 1}^{p} {S}_{τ}^{p} ⊗ {S}_{τ - 1}^{p} - \frac{{b}_{i - 1} ( u ) - 1}{λ} ,$$

where i denotes the trial number, u denotes the action (Go or No-go) and s^{p} contains posterior probabilities of different states under each policy p for time point r. Note that in the current implementation, the relevant transition is τ=3, because the transition between t=1 and t=2 does not depend on the agent's choices. π denotes posterior policy probabilities. Furthermore, to account for instrumental learning facilitated by Pavlovian responses (Dayan et al., 2006), here, policy-blending is used: the posterior probabilities of Pavlovian Go or No-go response are combined with instrumental Go and No-go policy probabilities, respectively, when updating beliefs about controlled state transitions.

The remaining two parameters η and λ in the above equation control the learning rate and the belief decay (unlearning) rate, respectively. Following the work of Sales et al. (2019), λ is assumed to depend on state-action prediction errors (SAPEs) and to be associated with effective connectivity from the dPFC 410 to the LC 406 (FIG. 4). The relationship between SAPEs and λ is modelled using a logistic function:

$$λ = {λ}_{min} + \frac{{λ}_{max} - {λ}_{min}}{1 + {e}^{g ( SAPE - m )}} ,$$

where g is the gradient, m is the midpoint, while λₘᵢₙ and λₘₐₓ are minimum and maximum function values. Note that higher SAPEs will result in a smaller λ, which will result in more belief decay because λ is a denominator in the updated equation. SAPE itself is defined as Kullback-Leibler (KL) divergence between BMA distributions at successive time steps:

$$SAPE ( t ) = {D}_{KL} [ ( {S}_{τ}^{t} ∥ {S}_{τ}^{t - 1} ) ] .$$

In the simulations presented in the present disclosure, which are merely illustrative and not limiting, SAPE is computed for t=3, after the action (Go/No-go) is performed and only for predictions about the final states (τ=3). BMAs themselves are computed via:

$${S}_{τ} = {\sum }_{p} {π}_{τ}^{p} \cdot {S}_{τ}^{p} ,$$

where π^{p} denotes posterior policy probabilities and s^{p} denotes posterior state probabilities for policy pττ at time point τ.

In addition to being sensitive to environmental change (i.e. volatility), the LC-NE system also coordinates aversive learning mediated by Amy-LC connectivity 402 (see FIG. 4) (Uematsu et al., 2017; Jacobs et al., 2020). To capture these effects, a learning rate dependency on outcome valence (assuming Amy 404 activation during aversive outcomes) is introduced, which is associated with the preference against aversive outcomes encoded in the C vector 610:

$$η = 1 + k \vert C ( o ) \vert ,$$

where C(o) is the value of prior preference for outcome o, with the parameterization being −c for the aversive stimulus outcomes and 0 for the neutral outcomes. Parameter k is a scaling factor that could correspond to effective connectivity 402 between the Amy 404 and the LC 406. Note that the learning rate dependence on valence introduced here is what enables the model to account for affective biases (Pulcu and Browning, 2017, 2019; Sharot and Garrett, 2016; Eshel and Roiser, 2010). A more principled implementation of valence and its role in modulating the learning rate could depend on the rate of change of free energy over time (Joffily and Coricelli, 2013).

The final component of the model aims to account for how controllability of aversive outcomes inhibits Amy 404 activation via the serotonergic system involving vmPFC-DRN-Amy network (404, 416, 418, 420, 422) (Maier and Seligman, 2016; Kerr et al., 2012). This is implemented within stress reactivity by modulating stress sensitivity parameter c by a controllability parameter w:

$${c}^{′} = {c}^{( 1 - w )}$$

In the limiting cases when there is no control (w=0), c′ is equal to the original c and when there is complete control (w=1) c′ is equal to 1. Controllability itself is assumed to depend on the mean of beliefs that the neutral outcome will be reached (i.e. based on state transition probabilities encoded in ACC 414) averaging across the two possible actions:

$${w}_{n} = \frac{1}{\vert a \vert} {Σ}_{a} P ( {o}_{6} ⋃ {o}_{8} \vert {s}_{n} , a ) , n ∈ [ 5 , 6 , 7 , 8 ]$$

Where |a| denotes the number of available actions, P (osn, a) is simply the product of the likelihood of observations A (602) and the state transitions B{Go} (608), B{Nogo} (606), B₀ (604) and n denotes the four available states at t=2, which correspond to the four different conditions. Parameter wₙ effectively represents average probability of achieving the desired outcome associated with each condition and thus with each cue. Note that this is similar to vmPFC 418 encoding expected value (see (Hiser and Koenigs, 2018) for a review). Furthermore, such distinction between vmPFC 418, which encodes expected outcome (which is associated with controllability), and ACC 414, which encodes state-transition probabilities (which is related to hopelessness), is consistent with the finding that vmPFC 418 encodes stimulus-based value and is more active during the outcome phase (cf. stress response) and that ACC 414 encodes action-based value and is more active during both outcome and decision phases (cf. instrumental control and learning) (Vassena et al., 2014). The close relationship between the subjective feeling of control and outcome valuation has also been demonstrated in recent studies (Stolz et al., 2020; Wang and Delgado, 2019). Relevantly, STB has been associated with reduced activation to expected value in vmPFC 418 (Brown et al., 2020; Dombrovski and Hallquist, 2017).

Finally, to collectively account for any impairments of how w modulates the stress response (i.e., any impairments along the vmPFC-DRN-Amy network), wₙ is transformed into the final estimate of controllability by entering it into a logistic function constrained by a controllability threshold w₀ (i.e. the midpoint of the logistic function) and a gradient g_{w}:

$$w = \frac{1}{1 + {e}^{- {g}_{w} ( {w}_{B} - {w}_{0} )}} .$$

FIG. 4A shows hypothesized brain network 400 of FIG. 4, with the proposed computations, possible neural correlates and parameters of interest 428 of a cognitive component 426 of the model (e.g. model 136) for STB as described above. There are four areas of relevance for the cognitive component 426 of the model: learning rate 430, belief decay rate 432, stress reactivity 434 and perceived stressor controllability 436. A stress weight parameter, k, controls the boost in the learning rate 430 in response to stress. Increasing this parameter would result in increased learning from stressful outcomes. A stress sensitivity parameter, c, captures individual sensitivity to stress, which then also affects the learning rate 430. A controllability threshold, w₀, is a midpoint in the logistic function that translates the beliefs about state transitions into an estimate of stressor controllability 436. In other words, w₀ regulates how positive state transition beliefs have to be for a stressor to be deemed sufficiently controllable. Finally, a belief decay threshold, m, regulates how large state-action prediction errors (SAPEs) have to be before significant belief decay (unlearning) takes place. Note that for the belief decay rate 432 and the stressor controllability 436 there are other parameters (gradients, g_{w}, g, and minimum and maximum decay values λₘᵢₙ, λₘₐₓ) that could be inspected, but for simplicity here the present disclosure focuses on the midpoint values w₀ and m as the exact parameterization of these effects is somewhat arbitrary and the midpoints are sufficient for exploring the general direction of different manipulations.

The simulation results presented below do not hinge on the additional computation associated with stressor controllability 436, except for the results concerning the parameter w for stressor controllability 436 itself.

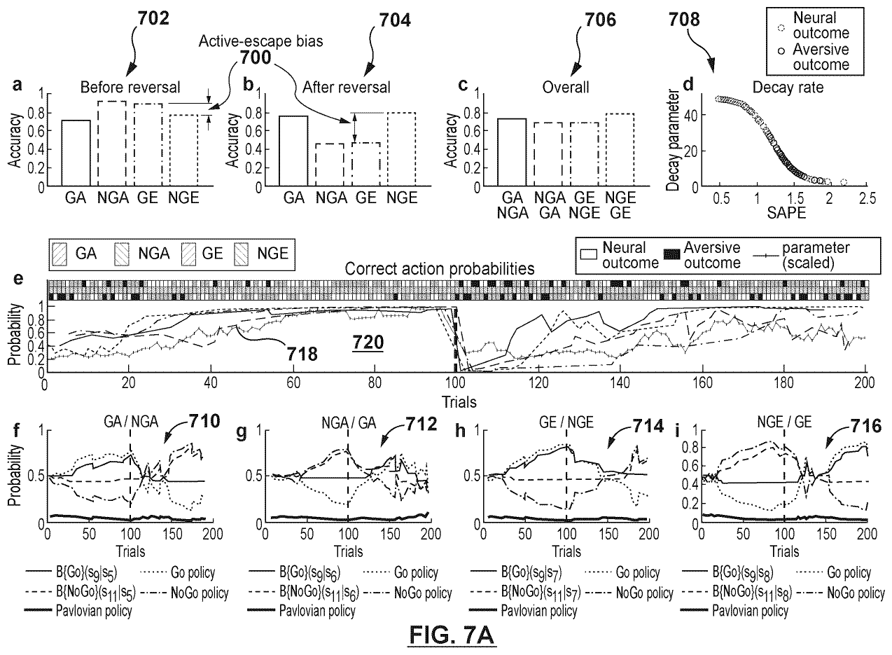

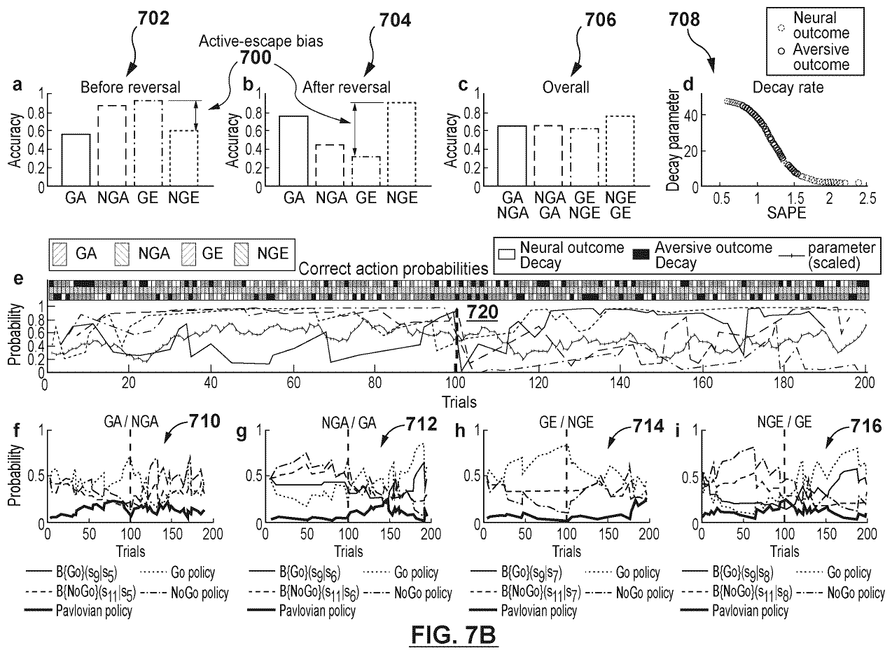

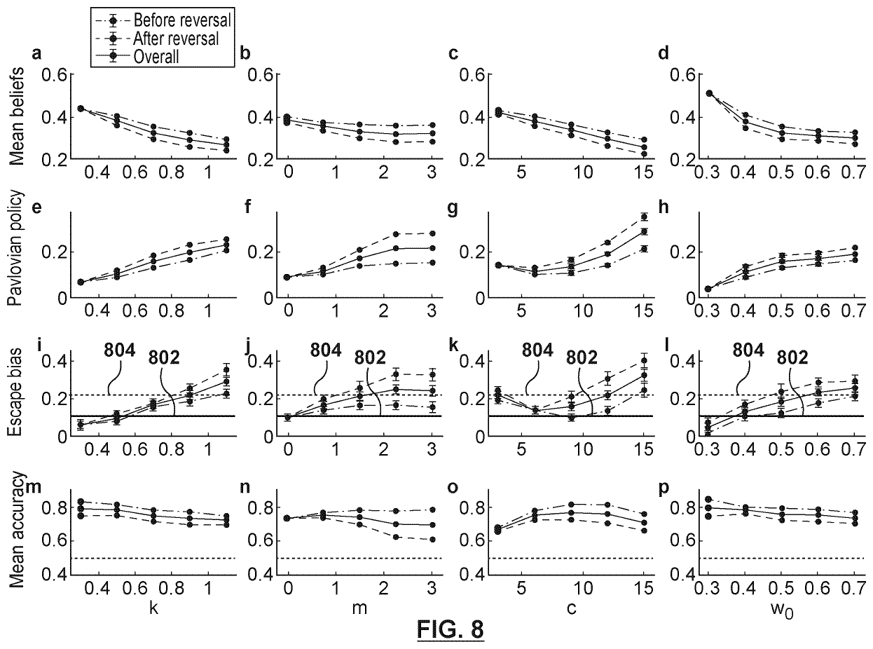

First, performance on the task shown in FIG. 5 was simulated for a single healthy control (see FIG. 7A). Increasing stress weight parameter k-which regulates the aversiveness-related component of the learning rate 430 and is presumably represented in terms of Amy-LC connectivity 402—can produce increased active-escape biases and other behavioral and cognitive aspects associated with suicidality (see FIG. 7B). Finally, a wider hypothesis space was defined, exploring how different parameters of interest 428 can independently lead to the behavior observed in STB (see FIG. 8).

Reference is first made to FIG. 7A. For the initial simulations 200 trials of the task shown in FIG. 5 were run, where at every trial one of the four cues was presented at random. After 100 trials, the meanings of the cues were reversed (i.e. a reversal as described above). In this simulation, the relevant variables were set to k=0.1, m=1.3, c=8, w₀=0.5, z=0.4, λₘᵢₙ=2, λₘₐₓ=50, α=3 and β=1 to produce reasonable performance trajectories as well as an active-escape bias 700 before reversal consistent with empirical findings reported by Millner et al. (2018). Accuracy for Go-to-Avoid (GA), No-Go-to-Avoid (NGA), Go-to-Escape (GE) and No-Go-to-Escape (NGE) is shown at 702 before the reversal, at 704 after the reversal, and overall at 706. The decay parameter values for different SAPEs throughout the task are shown at 708. Note that SAPEs for aversive outcomes are larger which leads to smaller decay parameter, and thus to larger belief decay. As the agent's beliefs approach the actual state transition probabilities, this makes the neutral outcomes more expected, thus invoking only small SAPEs in contrast to unexpected aversive outcomes. This is also what drives advantageous belief decay (successful unlearning) after the reversal: a series of negative outcomes with large SAPEs result in a sharp drop in the decay parameter (718, black line), which increases belief decay and facilitates quick learning of new contingencies. Correct action probabilities are shown at 720. The top 3-row panel shows the sequence of cue presentation (middle row), executed action (non-grey squares: bottom row—No-go, top row—Go) and trial outcome (white—neutral, black—aversive); each column corresponds to a single trial. The main panel shows trajectories of correct action probabilities, which gradually increase as the task progresses, but drop sharply once the Go/No-go cue meanings are reversed on the 100^{th} trial. The response to this environmental change can be seen in the decreased decay parameter (718, black line), which drives faster forgetting of previously learned contingencies and allows the agent to adapt. Note that decay parameter trajectory here is scaled to be between 0 and 1 and smoothed out using moving average with a window size of 5 trials. Trajectories of underlying beliefs about state transitions and policy probabilities are shown at 710 for Go-to-Avoid (GA)/No-Go-to-Avoid (NGA), at 712 for No-Go-to-Avoid (NGA)/Go-to-Avoid (GA), at 714 for Go-to-Escape (GE)/No-Go-to-Escape (NGE) and at 716 for No-Go-to-Escape (NGE)/Go-to-Escape (GE). These plots reflect the straightforward relationship between belief strength and policy probability: as the probability of an instrumental Go/No-go action leading to the desired state increases (solid/dash-dotted colored lines) the probability of choosing Go/No-go policy tracks that increase (solid/dash-dotted gray), and probabilities of Pavlovian policies (solid black, lowermost line) decrease as a result. The vertical dashed lines in all of the plots denote the reversal. The Pavlovian policy that underlies the active-escape bias can be seen at its strongest at the very beginning of the task and right after the reversal, when beliefs that instrumental actions will lead to neutral outcomes are lower (710, 712, 714, 716).

Reference is now made to FIG. 7B, which shows the same plots as FIG. 7A, with like references denoting like features, where parameter k is increased to 1. Average choice accuracy is shown before reversal at 702, after reversal at 704 and overall at 706, for Go-to-Avoid (GA), No-Go-to-Avoid (NGA), Go-to-Escape (GE) and No-Go-to-Escape (NGE). The results before reversal at 702 reproduce increased active-escape bias in suicidality reported by Millner et al. (2019), and predict that this bias would be even larger after a reversal as shown at 704. Decay parameter values for different SAPEs throughout the task are shown at 708. Note that now aversive outcomes produce smaller SAPEs, due to increased expectation of aversive states. Performance across all trials is shown at 720. The top 3-row panel shows the sequence of cue presentation (middle row), executed action (non-grey squares: bottom row-No-go, top row—Go) and trial outcome (white-neutral, black-aversive); each column corresponds to a single trial. The main panel shows trajectories of correct action probabilities. Compared to the healthy control in the previous figure, the trajectories are noisier, especially after the reversal on the 100^{th} trial. Decay rate trajectory (718, black line) is also nosier, which is partly responsible for the poor adaptation after the reversal. Note that decay parameter trajectory here is scaled to be between 0 and 1 and smoothed out using moving average with a window size of 5 trials. Trajectories of underlying beliefs about state transitions and policy probabilities are shown at 710 for Go-to-Avoid (GA)/No-Go-to-Avoid (NGA), at 712 for No-Go-to-Avoid (NGA)/Go-to-Avoid (GA), at 714 for Go-to-Escape (GE)/No-Go-to-Escape (NGE) and at 716 for No-Go-to-Escape (NGE)/Go-to-Escape (GE). Compared to the healthy control (FIG. 7A), the belief trajectories are noisier, but even more importantly, beliefs about the instrumental transitions to neutral states are on average weaker (cf. hopelessness), which leads to increased probability of the Pavlovian policy. The vertical dashed lines in all of the plots denote the reversal.

By increasing parameter k to 1, the size of the belief update after experiencing aversive outcomes becomes larger, reproducing the increased active-escape bias 700 by a similar magnitude as reported in individuals with STB (Millner et al., 2019). The increase in the active-escape bias 700 is a direct consequence of the increased influence of the Pavlovian policy (710, 712, 714, 716, solid black, lowermost line), which in turn is a consequence of weaker beliefs that either of the instrumental Go/No-Go actions will lead to the desired neutral outcome (cf. hopelessness) (710, 712, 714, 716, colored lines). The latter is a direct consequence of increased k, leading to an over-adjustment of beliefs after aversive outcomes. This also disrupts the agent's ability to adapt to a changing environment because negative outcomes after the reversal become less surprising: this is reflected in reduced SAPEs for aversive outcomes and increased SAPEs for neutral outcomes as shown at 708. Assuming SAPEs are computed in dPFC 410 (Sales et al., 2019; Gläscher et al., 2010), this result would be consistent with empirical findings of increased dPFC 410 response to wins vs. losses in suicide attempters (Olić et al., 2015) and 341 reduced dlPFC activation in response to negative stimuli in suicidal ideation (Miller et al., 2018).

While directly increasing learning from aversive outcomes (k) is one way to produce the effects associated with STB, there is a wider hypothesis space to be explored. To that end, a more extensive investigation of the effects of other model parameters was performed. In this context, it is important to note that the model exhibits a considerable degree of stochasticity when initiated with the chosen parameter configurations and thus, the results presented earlier in FIGS. 7A and 7B are meant to be primarily illustrative, and are not intended to be limiting. To reduce stochasticity and to obtain more robust behavioral results, 400 trials (with a reversal at 200) were used, running 30 simulations for each parameter configuration. To visualize the results, relevant task performance summary statistics (mean and standard error) were computed for each parameter configuration, as shown in FIG. 8. Each column shows the effects of varying the parameters. The leftmost column shows the effect of varying k—stress weight, the second column from the left shows the effect of varying m—belief decay threshold, the third column from the left (second from right) shows the effect of varying c—stress sensitivity and the rightmost column shows the effect of varying w₀—controllability threshold. The initial settings of parameters were: k=0.6, m=1.2, c=8, w₀=0.6, z=0.3. The top row shows the mean of beliefs that the neutral state will be reached averaged across 4 contexts and 2 possible actions. The second row from the top shows the mean probability of choosing the Pavlovian policy. The third row from the top (second from the bottom) shows active-escape bias (the difference between choice accuracy on GE and NGE trials). The solid lines 802 and dashed lines 804 denote the expected active-escape bias in healthy control group and suicidality group, respectively (based on Millner and colleagues findings (Millner et al., 2018, 2019)). The bottom row shows mean choice accuracy across all 4 contexts.

The first (leftmost) column in FIG. 8 reproduces the results in FIGS. 7A and 7B, showing that increasing learning from negative outcomes reduces beliefs that instrumental actions will lead to the desired states (top row), which leads to an increase in the probability of the Pavlovian policy (second row from top), which in turn leads to a larger active-escape bias (third row from top, second row from bottom). As a result of the increased biases, a slight decrease in the overall performance accuracy is observed (bottom row).

The second column from the left in FIG. 8 shows that reducing base belief decay (increasing parameter m) produces similar results of more negative beliefs (top row), a higher probability of the Pavlovian policy (second row from top) and a stronger active-escape bias (third row from top, second row from bottom). A deterioration of the overall performance accuracy after the reversal is also observed (bottom row), as the agent is slow to adapt to new contingencies. Although very little research exists on reversal learning in suicidality, the latter result is in line with impaired reversal learning demonstrated in a reward/punishment probabilistic learning task in suicide attempters (Dombrovski et al., 2010).

As shown in the third column from the left (second from right) in FIG. 8, a higher stress sensitivity (larger c) also produces the effects associated with STB: more negative beliefs (top row) lead to a higher probability of the Pavlovian policy (second row from top) and a stronger active-escape bias (third row from top, second row from bottom). Finally, the overall performance accuracy (bottom row) shows a non-linear dependence on stress sensitivity c, which is reminiscent of the inverted U-shaped relationship between stress and performance (Yerkes et al., 1908; Hebb, 1955). The c parameter features in the model twice: first, in the prior over outcomes, and second, in the learning rate after aversive outcomes. The decrease in the overall performance accuracy and the increase in the active-escape bias at very low values of c can be explained by the former role of this parameter. In other words, a small c means little motivation to prefer neutral outcomes (e.g., the aversive outcomes are not experienced as very aversive), which leads to a more random policy selection and thus effectively increases Pavlovian influences and reduces overall performance accuracy. In contrast, the increased active-escape bias associated with larger c values derives from parameter c's contribution to the learning rate. Interestingly, both reduced and increased distress tolerance have been associated with suicide risk: lower distress tolerance relates to psychological/social pain and contributes to suicidal ideation, while higher distress tolerance relates to physical pain and contributes to the acquired capability for suicide (see (Liu et al., 2016) for discussion). The presently described model simulations are agnostic to the nature of the aversive stimulus used and thus might be capturing both of these effects.

Turning to the rightmost column in FIG. 8, reducing perceived controllability (increasing w₀) is yet another way to produce the effects associated with STB. By way of a self-fulfilling prophesy, a reduced controllability threshold leads to more negative beliefs (top row), which induces increases in the Pavlovian policy probability (second row from top) and an active-escape bias (third row from top), as well as a slight decrease in the overall performance accuracy (bottom row).

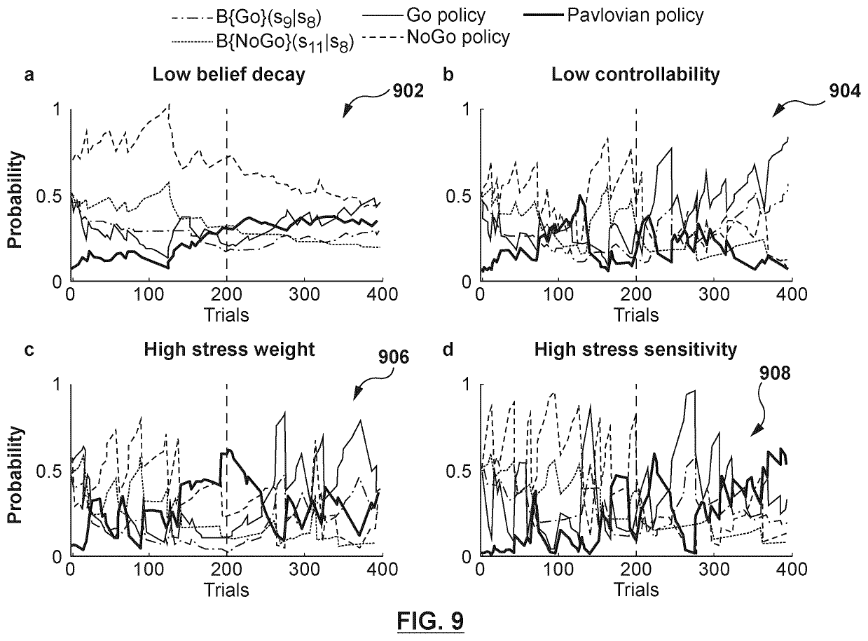

While all of the above parameter manipulations lead to similar mean behavioral effects, inspecting the time series reveals different dynamics of belief updating and policy probabilities, as shown in FIG. 9. FIG. 9 shows low belief decay, m=3 at reference 902, low controllability, w₀=1 at 904, high stress weight, k=1.2 at 906 and high stress sensitivity, c=14 at 908. The initial settings of parameters were: k=0.6, m=1.2, c=8, w₀=0.6, z=0.3. All panels show trajectories of NGE/GE cue: where the cue is NGE before the reversal (the vertical dashed line) and GE after the reversal. Less variable rigid negative beliefs and Pavlovian policy at 902 could be associated with planful suicide attempts, whereas more variable beliefs and sudden increases in Pavlovian policy at 904, 906 and 908 could be associated with more impulsive suicide attempts (Schmaal et al., 2020; Bernanke et al., 2017). Using NGE/GE cue as an example, for high m values (low belief decay rate 902), a very gradual progression towards more negative beliefs and an increased influence of the Pavlovian policy is observed. For high w₀ (low controllability 904), high k (high stress weight 906) and high c (high stress sensitivity 908), increasingly larger and sudden spikes in Pavlovian biases are seen. Considering the influence of the Pavlovian policy as a proxy for STB risk, the former scenario suggests a constantly increasing risk of STB and thus could be related to planful suicide attempts, while the latter scenario suggests an increased STB risk immediately after the occurrence of aversive events and could relate to impulsive suicide attempts. Bearing in mind the proposed links between the model parameters 428, 430, 432 and the underlying neurocircuitry of the brain network 400 (FIG. 4A) these results are consistent with planful and impulsive suicide attempt subtypes, with the former being predominantly associated with dPFC 410 activity and the latter being predominantly associated with vmPFC 418 activity (Schmaal et al., 2020).

The foregoing description presents a computational model of hopelessness and Pavlovian/active-escape bias in suicidality. This model shows that increased Pavlovian control and active-escape biases result from state hopelessness via the drive to maximize model evidence. Moreover, the foregoing description proposes how hopelessness itself can arise from four mechanisms: (1) increased learning from aversive outcomes, (2) reduced belief decay in response to unexpected outcomes, (3) increased stress sensitivity c, and (4) reduced sense of stressor controllability, and how these alterations might relate to the neurocircuits implicated in suicidality. Specifically, perturbations in the LC-NE system were considered together with the Amy 404, the dPFC 410 and the ACC 414, which mediate learning in response to acute stress and volatility, as well as perturbations in the DRN-5-HT system together with the vmPFC 418 and the Amy 404, which regulate stress reactivity and its modulation by perceived controllability. The model was validated via simulations of an Avoid/Escape Go/No-go task reproducing the active-escape biases reported by Millner and colleagues (Millner et al., 2019, 2018).

Importantly, the proposed model described in the present disclosure provides advantages and new insights compared to previous modelling work. Millner et al. (2019) analyzed the increased active-escape bias in STB using a combined reinforcement learning-drift diffusion model (RL-DDM) and found that an increased active-escape bias can be explained by a bias parameter (aka a starting point in the DDM part of the model). This parameter was assumed to be constant throughout the task. In contrast, the proposed model described in the present disclosure offers a mechanistic explanation for how active-escape bias arises dynamically from learning about the state transition probabilities and balancing between instrumental and Pavlovian policies. Unlike in RL-DDM, in the model according to the present disclosure, Pavlovian and instrumental policies are represented explicitly. Importantly, this allows state transition probabilities to be related to state hopelessness (which is a central construct in suicidality research (Klonsky et al., 2018; May et al., 2020; Isometsä, 2014)), offering a possible operationalization of the hopelessness construct. Finally, using the active inference framework enabled the proposal of several links between the model parameters 428, 430, 432 and the underlying neurocircuitry of the brain network 400, which could help bridge the explanatory gap between neurobiology and cognition in STB.

The present model simulation results offer a computational hypothesis space by identifying mechanistically distinct perturbations that lead to hopelessness and Pavlovian/active-escape biases associated with STB. These distinct pathways might also speak to different suicidality subtypes: impulsive versus planful (Schmaal et al., 2020; Bernanke et al., 2017). While all of the four parameter manipulations produced increased Pavlovian control and active-escape biases, examining the trajectories of belief updating revealed that reduced belief decay led to more gradual updates and more stable negative beliefs as well as more stable and elevated Pavlovian influences, which could be associated with more planful STB. The other three manipulations reduced controllability of stressors, increased learning from aversive outcomes and increased stress sensitivity parameter c-resulted in increasingly variable belief updates with sudden spikes in Pavlovian biases after aversive outcomes, which could be associated with more impulsive STB. Referring to FIG. 4A, considering the dPFC 410 and the vmPFC 418 as possible correlates of belief decay 432 and controllability 436 (and its effects on stress reactivity 434), respectively, the results are in agreement with neuroimaging studies associating disruptions in vmPFC 418 activity with the impulsive STB subtype and the dPFC 410 activity with the planful STB subtype (Schmaal et al., 2020).

While the present disclosure adopts a transdiagnostic view of STB for purposes of explication, many mental disorders are known to increase suicide risk. Among all disorders, borderline personality disorder (BPD), depression, bipolar disorder, schizophrenia, and anorexia nervosa show the highest risk of suicide—between 10 to 45 times higher than the general population (Chesney et al., 2014). Comorbidities further increase suicide risk by inflicting higher levels of distress (Nock et al., 2010; Jylhä et al., 2016), with the majority of suicides being estimated to occur within a major depressive episode (Isometsä, 2014). Recent studies show preliminary evidence that suicide subtypes might cut across the current categories of disorders, with higher suicidal ideation variability (i.e. higher stress responsiveness) being associated with childhood physical abuse, aggression, and impulsivity in major depressive disorder (Oquendo et al., 2020) and with affective lability in BDP (Rizk et al., 2019). In a similar way, the ways in which different mental disorders increase the risk of suicide could also map onto the different ways in which the effects associated with STB can emerge within the presently described model.

Being able to stratify the propensity for suicidal behavior into mechanistically distinct subgroups could help improve early interventions and treatment response prediction. Many different psychotherapies are applied in the context of suicidality, including the manualized therapies such as CBT, Dialectical Behavior Therapy (DTB), and mentalization-based therapy (MTB). However, evidence for the effectiveness of different psychotherapies is still scarce and it remains unclear which components of the therapies are most effective in reducing suicidality (Briggs et al., 2019; Ougrin et al., 2015; Weinberg et al., 2010). Moreover, the attempts to determine these unknowns are likely complicated by not accounting for the etiological heterogeneity in high suicide risk groups (Iyengar et al., 2018). Current neurobiological models of the mechanism of action of psychotherapy point to neural substrates of executive and semantic processes and highlight the vmPFC 418 and its involvement in implicit emotion regulation as well as dPFC 410 and its involvement in explicit behavioral control (Messina et al., 2016). This would map to the stressor controllability 436 (vmPFC 418) and belief decay 432 (dPFC 410) components in the presently described model and would suggest these parameters to be relevant when assessing, monitoring or optimizing the effectiveness of psychotherapy for a given suicidality subtype. For example, the stressor controllability parameter 436 may be seen as reflecting the level of felt control over one's inner and outer life whereas the belief decay parameter could capture one's ability to unlearn maladaptive beliefs through new experiences, behavior or cognitive reappraisal (Zilverstand et al., 2017).

In applying pharmacotherapy to STB, sub-anesthetic doses of ketamine, a N-methyl-D-aspartate receptor (NMDAR) antagonist, is currently one of the most promising interventions for rapid reduction of STB, but only 55-60% of individuals respond with a complete remission (Wilkinson et al., 2018). The exact mechanism through which ketamine achieves its anti-suicidal and anti-depressant effects is still not fully understood (Riggs and Gould, 2021). Many hypotheses emphasize the importance of increased α-amino-3-hydroxy-5-methyl-4-isoxazolepropionic acid receptor (AMPAR) signaling, its involvement in bottom-up information transmission and a consequent increase in synaptic and spine plasticity (Zanos and Gould, 2018; Lengvenyte et al., 2019). Other recent in vivo microdialysis findings suggest ketamine-induced AMPAR signaling in LC and DRN as well as a subsequent release of NE and 5-HT in the mPFC to be necessary for the rapid antidepressant effects (López-Gil et al., 2019; Llamosas et al., 2019; Pham et al., 2017), also implicating prelimbic cortex (a homolog to Brodmann's area in the vmPFC 418) (PL)-DRN involvement in stressor controllability (Amat et al., 2016; Dolzani et al., 2018). A recent review also highlights the ACC 414 to be playing a key role in mediating ketamine's antidepressant effects (Alexander et al., 2021). The model described herein could help provide a more mechanistic understanding of how the changes in belief updating and possibly activity in these brain regions relate to reduced suicide risk.

Personalization of early interventions could also be improved by a more mechanistic understanding of sex differences as it relates to STB (Williams and Trainor, 2018). Females show a higher incidence of suicidal intent and suicide attempts, although the rate of completed suicides is much higher in males (2 to 5 times) (Freeman et al., 2017). Suicide risk factors have also been found to differ between the sexes (Oquendo et al., 2007). While multiple psychosocial factors are likely to be contributing to these differences (Canetto and Sakinofsky, 1998), sexual dimorphisms in the brain might play an important role as well (Pallayova et al., 2019). For example, structural and functional dimorphisms in the LC-NE system and its regulation by estrogen in females is associated with an increased susceptibility to hyperarousal (Bangasser ct al., 2016), which itself has been linked to a higher risk of suicidal ideation (Steyn et al., 2013; Morabito et al., 2020; Dolsen et al., 2017). Preclinical studies also suggest important sex differences in how stressor controllability modulates stress reactivity. Unlike males, females do not seem to benefit from increased controllability, with the lack of engagement and structural plasticity within the PL-DRN pathway being a likely mechanism for these differences (Fallon et al., 2020). The model described herein might help better understand how these differences impact stress reactivity and controllability, and how this affects response to ketamine as well as to other interventions (Fallon et al., 2020).

Thus, the apparatus 100 described above may be used for administering the method 200 for predicting active-escape bias in a mammalian subject to transform the physical signals according to a predefined model 136, 426, 600 to obtain at least one learning variable of the mammalian subject, and apply the predefined model to the learning variable(s) to classify an expected cause of an individual bias of the mammalian subject toward or away from active-escape behaviour. The learning variable(s) may be one or both of a belief decay rate 432 of the mammalian subject and a learning rate 430 of the mammalian subject, and may also include a stress sensitivity parameter c for the mammalian subject and/or a controllability threshold parameter w₀ for the mammalian subject.

As can be seen from the above description, the method for predicting active-escape bias in a mammalian subject described herein represents significantly more than merely using categories to organize, store and transmit information and organizing information through mathematical correlations. Importantly, no claim is made to any mathematical formulae, natural phenomena or laws of nature. The method for predicting active-escape bias in a mammalian subject transforms physical signals, and in particular simple “Go/No-go” physical signals, according to a predefined model to obtain at least one learning variable of the mammalian subject and applies the predefined model to the learning variable(s) to classify an expected cause of an individual bias of the mammalian subject toward or away from active-escape behaviour. As such, there is an improvement to a specific field of technology, namely, neurological analysis. Moreover, the method for predicting active-escape bias in a mammalian subject is applied by using a particular machine, namely an apparatus that comprises a physical cue device, a physical stimulator and a physical actuator, all coupled to a control device, which cooperate to administer cues and physical stimuli to, and receive physical signals from, a mammalian subject, all according to a predetermined pattern. Thus, there are physical elements which perform physical steps according to a predetermined pattern, and therefore manifest a discernible physical effect or change, as well as physical elements that receive physical inputs from the physical world, which inputs are then transformed according to a predefined model to obtain actionable diagnostic information. By transforming physical signals according to a task-specific model of neurochemically mediated cognitive processes, the method provides digital information that is representative of these underlying neurochemically mediated cognitive processes within a mammalian brain. Thus, implementation of the method using a specific machine (e.g. apparatus 100) is an analysis of digital signals of an underlying biological process, analogous to implementation of algorithmic analysis of digital signals from, for example, and ECG or MRI device to produce data for use in supporting a medical practitioner. Of note, the present method does not produce a diagnosis (e.g. of STB), but rather provides diagnostic information that can be used by a medical professional to make a diagnosis using their professional skill and judgment, much as a cardiologist might make a diagnosis using an ECG readout or a radiologist might do the same with an MRI image. The present disclosure describes a specific process for obtaining digital signals and transforming them to obtain specific information about specific neurological processes.

The foregoing discussion considers certain particular neurocircuits and neuromodulatory systems at the overlap of stress response, aversive learning, behavioral control and STB, but this is not intended to be limiting. There remain other relevant regions to be considered (Schmaal et al., 2020; Lengvenyte et al., 2019), and it is contemplated that methods according to the present disclosure may be applied in respect of some such regions. For example, one such region to consider may be the lateral habenula (LHb), an epithalamic nucleus acting as a relay hub between forebrain and midbrain structures and playing a significant role in learning from non-rewarding and aversive experiences (Matsumoto and Hikosaka, 2009). The LHb is involved in stressor controllability effects via the DRN-5-HT system (Metzger et al., 2017) and is one of the locations targeted by ketamine that mediates anti-depressant effects (Zanos and Gould, 2018; Yang et al., 2018a; Shepard et al., 2018). LHb activity has been associated with depressive symptoms of helplessness, anhedonia, and excessive negative focus (Yang et al., 2018b), while a recent study also reported higher resting state functional connectivity between LHb and several brain regions, including the amygdala, to be associated with STB independently of depressive symptoms (Ambrosi et al., 2019).

While a close consideration of the networks implicated in STB informed the construction of the illustrative model 136 (comprising task component 600 and cognitive component 426) described herein, implementation of the model is not at the level of neural dynamics but rather at the level of higher-order computational mechanisms underwritten by such dynamics (cf. Marr's levels of analysis (Marr and Poggio, 1976)). This means that the model variables might not necessarily neatly map onto distinct elements of the neurocircuitry but might interact with several other factors. For example, while stress sensitivity parameter c in the prior over outcomes is regarded as corresponding to stress sensitivity and Amy 404 activation, other factors may contribute to dispreference of the aversive outcome beyond its aversiveness per se, such as contextual factors relating to task engagement and a general motivation to do well in the task. Similarly, the controllability threshold w₀ might reflect a combined influence of changes in vmPFC 418 activation, its connectivity to the DRN 420, connectivity from the DRN 420 to the Amy 404 or even the LHb and the effects it exerts on the DRN-5-HT system.

Accordingly, the present disclosure is not intended to be exhaustive. The emergence of STB risk factors in different contexts is most likely to involve additional variables. Furthermore, the simulations explored only the simplest scenarios of varying one parameter at a time. Considering how these parameters interact provides another layer of complexity. For example, different subtypes of STB may be related not to a single parameter, but to a unique combination of multiple parameters, forming distinct clusters within the multidimensional parameter space. Future work with empirical data will allow for the further refinement of the model 136 and the delineation of different STB subtypes.

As noted above, the present method is applied to using a particular machine (e.g. apparatus 100), and as such the model 136 and the information generated by transforming the digital signals received by the machine is limited by the behavioral task 500 for which the machine is configured and around which the task component 600 of the model 136 is defined. In particular, in the task 500 considered here, the stimulus is completely unambiguous and there is only one decision per trial to make. Introducing sensory uncertainty and multiple decisions—which is when the active inference framework can be utilized more fully-would provide a richer context to study learning and behavior. Such tasks would allow for the capture of other phenomena relevant for STB, for example aversive generalization (how specific aversive events lead to negative beliefs about the world), its relationship to trauma, its effects on reduced problem-solving abilities (i.e. planning) and its influence on biases towards escape strategies (Linson and Friston, 2019; Linson et al., 2020).

The present disclosure does not explicitly address the distinction between suicide ideators and suicide attempters. Recent accounts of suicidality argue that suicidal ideation and the progression from ideation to attempts should be treated as separate processes (Van Orden et al., 2010; Klonsky and May 2015; O'Connor and Kirtley, 2018; Bryan et al., 2020; Klonsky et al., 2018). The active inference framework, and in particular classification of an expected cause of an individual bias of a subject toward or away from active-escape behaviour as enabled by the present disclosure, might be well suited to study these distinctions as the active inference framework explicitly models and factorizes inferences about the states of the world (cf. suicidal ideation) and action selection (cf. suicide attempt).

The present technology may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology have been described above with reference to flowchart illustrations and/or block diagrams of methods, apparatus (systems) and computer program products according to various embodiments. In this regard, the flowchart and block diagrams in the Figures illustrate the architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, other programmable data processing apparatus, or other devices to function in a particular manner, such that the instructions stored in the computer readable storage medium produce an article of manufacture including instructions which implement aspects of the functions/acts specified in the flowchart and/or block diagram block or blocks. The computer readable program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

As noted above, the control device 118 may be, for example, a suitably programmed general purpose computer, including any of a desktop computer, laptop computer, tablet computer, or smartphone, among others.

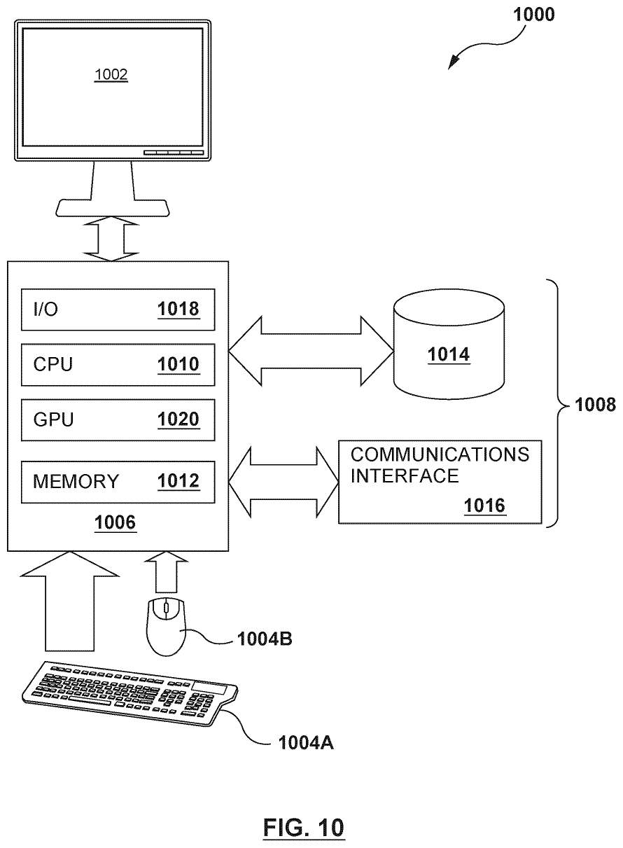

An illustrative computer system in respect of which the technology herein described may be implemented is presented as a block diagram in FIG. 10. The illustrative computer system is denoted generally by reference numeral 1000 and includes a display 1002, input devices in the form of keyboard 1004A and pointing device 1004B, computer 1006 and external devices 1008. While pointing device 1004B is depicted as a mouse, it will be appreciated that other types of pointing device, or a touch screen, may also be used.

The computer 1006 may contain one or more processors or microprocessors, such as a central processing unit (CPU) 1010. The CPU 1010 performs arithmetic calculations and control functions to execute software stored in an internal memory 1012, preferably random access memory (RAM) and/or read only memory (ROM), and possibly additional memory 1014. The additional memory 1014 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 1014 may be physically internal to the computer 1006, or external as shown in FIG. 10, or both.

The computer system 1000 may also include other similar means for allowing computer programs or other instructions to be loaded. Such means can include, for example, a communications interface 1016 which allows software and data to be transferred between the computer system 1000 and external systems and networks. Examples of communications interface 1016 can include a modem, a network interface such as an Ethernet card, a wireless communication interface, or a serial or parallel communications port. Software and data transferred via communications interface 1016 are in the form of signals which can be electronic, acoustic, electromagnetic, optical or other signals capable of being received by communications interface 1016. Multiple interfaces, of course, can be provided on a single computer system 1000.

Input and output to and from the computer 1006 is administered by the input/output (I/O) interface 1018. This I/O interface 1018 administers control of the display 1002, keyboard 1004A, external devices 1008 and other such components of the computer system 1000. The computer 1006 also includes a graphical processing unit (GPU) 1020. The latter may also be used for computational purposes as an adjunct to, or instead of, the (CPU) 1010, for mathematical calculations.

The various components of the computer system 1000 are coupled to one another either directly or by coupling to suitable buses.

In some embodiments, a computer such as the computer 1000 may comprise the entirety of an apparatus 100 (FIG. 1). The display 1002 and/or an inbuilt or peripheral speaker may serve as a cue device 112, the speaker may be used as a physical stimulator 114, and the keyboard 1104A, mouse 1104B or other input device may serve as a physical actuator 116. The computer system 1006, optionally in conjunction with additional memory 1014, may function as control apparatus 118.

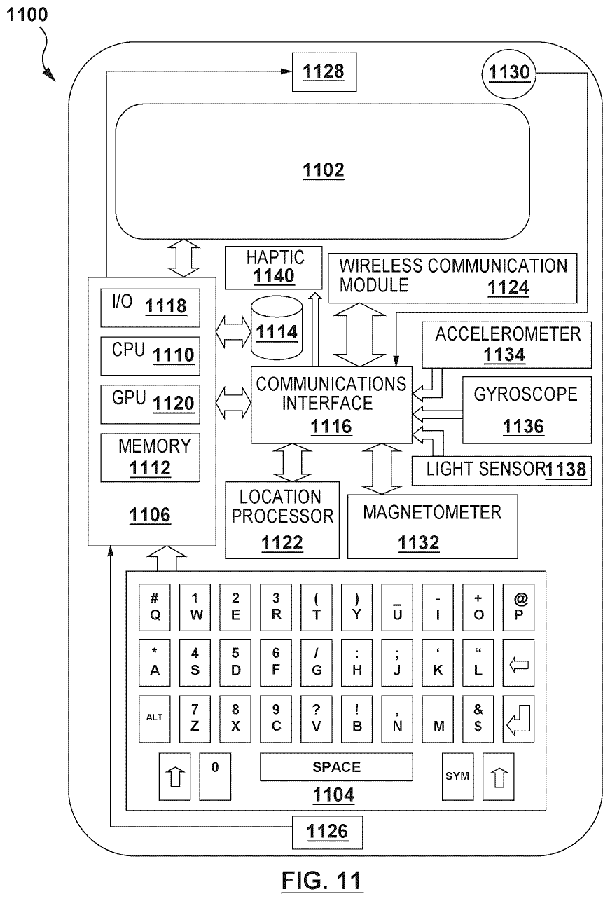

FIG. 11 shows an illustrative networked mobile wireless telecommunication computing device in the form of a smartphone 1100. The smartphone 1100 includes a display 1102, an input device in the form of keyboard 1104 and an onboard computer system 1106. The display 1102 may be a touchscreen display and thereby serve as an additional input device, or as an alternative to the keyboard 1104. The onboard computer system 1106 comprises a central processing unit (CPU) 1110 having one or more processors or microprocessors for performing arithmetic calculations and control functions to execute software stored in an internal memory 1112, preferably random access memory (RAM) and/or read only memory (ROM) is coupled to additional memory 1114 which will typically comprise flash memory, which may be integrated into the smartphone 1100 or may comprise a removable flash card, or both. The smartphone 1100 also includes a communications interface 1116 which allows software and data to be transferred between the smartphone 1100 and external systems and networks. The communications interface 1116 is coupled to one or more wireless communication modules 1124, which will typically comprise a wireless radio for connecting to one or more of a cellular network, a wireless digital network or a Wi-Fi network. The communications interface 1116 will also typically enable a wired connection of the smartphone 1100 to an external computer system. A microphone 1126 and speaker 1128 are coupled to the onboard computer system 1106 to support the telephone functions and other functions managed by the onboard computer system 1106, and a location processor 1122 (e.g. including GPS receiver hardware) may also be coupled to the communications interface 1116 to support navigation operations by the onboard computer system 1106. One or more cameras 1130 (e.g. front-facing and/or rear facing cameras) may also be coupled to the onboard computer system 1106, as may be one or more of a magnetometer 1132, accelerometer 1134, gyroscope 1136 and light sensor 1138. The smartphone 1100 may also include haptic feedback hardware 1140 coupled to the onboard computer system 1106. Input and output to and from the onboard computer system 1106 is administered by the input/output (I/O) interface 1118, which administers control of the display 1102, keyboard 1104, microphone 1126, speaker 1128, camera 1130, magnetometer 1132, accelerometer 1134, gyroscope 1136 and light sensor 1138. The onboard computer system 1106 may also include a separate graphical processing unit (GPU) 1120. The various components are coupled to one another either directly or by coupling to suitable buses.

In some embodiments, a smartphone such as the smartphone 1100 may comprise the entirety of an apparatus 100 (FIG. 1). The display 1102 and/or speaker 1128 may serve as a cue device 112, the haptic feedback hardware 1140 and/or the speaker 1128 may be used as a physical stimulator 114, and the keyboard 1104 and/or touchscreen display and/or other button(s) may serve as a physical actuator 116. The onboard computer system 1106, possibly in conjunction with additional memory 1114, may function as control apparatus 118.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 1112 of the onboard computer system 1106 of the smartphone 1100 or the memory 1012 of the computer 1006, or on a computer usable or computer readable medium external to the onboard computer system 1106 of the smartphone 1100 or the computer 1006, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

One or more currently preferred embodiments have been described by way of example. It will be apparent to persons skilled in the art that a number of variations and modifications can be made without departing from the scope of the claims.

The following list of references is provided for the reader's convenience, without any admission that any of the references constitute prior art citable against the present application, and without any admission that any of the references are relevant to the invention as claimed:
Akam, T., Rodrigues-Vaz, I., Marcelo, I., Zhang, X., Pereira, M., Oliveira, R. F., Dayan, P., and Costa, R. M. (2021). The anterior cingulate cortex predicts future states to mediate model-based action selection. Neuron, 109(1):149-163.
Alarcón, G., Sauder, M., Teoh, J. Y., Forbes, E. E., and Quevedo, K. (2019). Amygdala functional connectivity during self-face processing in depressed adolescents with recent suicide attempt. Journal of the American Academy of Child & Adolescent Psychiatry, 58(2):221-231.
Alexander, L., Jelen, L. A., Mehta, M. A., and Young, A. H. (2021). The anterior cingulate cortex as a key locus of ketamine's antidepressant action. Neuroscience Biobehavioral Reviews.
Amat, J., Dolzani, S. D., Tilden, S., Christianson, J. P., Kubala, K. H., Bartholomay, K., Sperr, K., Ciancio, N., Watkins, L. R., and Maier, S. F. (2016). Previous ketamine produces an enduring blockade of neurochemical and behavioral effects of uncontrollable stress. Journal of Neuroscience, 36(1):153-161.
Ambrosi, E., Arciniegas, D. B., Curtis, K. N., Patriquin, M. A., Spalletta, G., Sani, G., Frueh, B. C., Fowler,
J. C., Madan, A., and Salas, R. (2019). Resting-state functional connectivity of the habenula in mood disorder patients with and without suicide-related behaviors. The Journal of neuropsychiatry and clinical neurosciences, 31(1):49-56.
Arango, V., Underwood, M. D., Boldrini, M., Tamir, H., Kassir, S. A., Hsiung, S. c., Chen, J. J., and Mann, J. J. (2001). Serotonin 1a receptors, serotonin transporter binding and serotonin transporter mrna expression in the brainstem of depressed suicide victims. Neuropsychopharmacology, 25(6):892-903.
Bach, H., Huang, Y. Y., Underwood, M. D., Dwork, A. J., Mann, J. J., and Arango, V. (2014). Elevated serotonin and 5-hiaa in the brainstem and lower serotonin turnover in the prefrontal cortex of suicides. Synapse, 68(3): 127-130.
Baek, K., Kwon, J., Chae, J. H., Chung, Y. A., Kralik, J. D., Min, J. A., Huh, H., Choi, K. M., Jang, K. I., Lee, N. B., et al. (2017). Heightened aversion to risk and loss in depressed patients with a suicide attempt history. Scientific reports, 7(1): 1-13.
Balcioglu, Y. H. and Kose, S. (2018). Neural substrates of suicide and suicidal behaviour: from a neuroimaging perspective. Psychiatry and Clinical Psychopharmacology, 28(3):314-328.
Bangasser, D. A., Wiersielis, K. R., and Khantsis, S. (2016). Sex differences in the locus coeruleus-norepinephrine system and its regulation by stress. Brain research, 1641:177-188.
Baumeister, R. F. (1990). Suicide as escape from self. Psychological review, 97(1):90.
Bernanke, J., Stanley, B., and Oquendo, M. (2017). Toward fine-grained phenotyping of suicidal behavior: the role of suicidal subtypes. Molecular psychiatry, 22(8): 1080-1081.
Bouret, S. and Sara, S. J. (2005). Network reset: a simplified overarching theory of locus coeruleus noradrenaline function. Trends in neurosciences, 28(11):574-582.
Bridge, J. A., Reynolds, B., McBee-Strayer, S. M., Sheftall, A. H., Ackerman, J., Stevens, J., Mendoza, K., Campo, J. V., and Brent, D. A. (2015). Impulsive aggression, delay discounting, and adolescent suicide attempts: effects of current psychotropic medication use and family history of suicidal behavior. Journal of child and adolescent psychopharmacology, 25(2): 114-123.
Briggs, S., Netuveli, G., Gould, N., Gkaravella, A., Gluckman, N. S., Kangogyere, P., Farr, R., Goldblatt,
M. J., and Lindner, R. (2019). The effectiveness of psychoanalytic/psychodynamic psychotherapy for reducing suicide attempts and self-harm: systematic review and meta-analysis. The British Journal of Psychiatry, 214(6):320-328.
Brown, V. M., Wilson, J., Hallquist, M. N., Szanto, K., and Dombrovski, A. Y. (2020). Ventromedial prefrontal value signals and functional connectivity during decision-making in suicidal behavior and impulsivity. Neuropsychopharmacology, 45(6): 1034-1041.
Bryan, C. J., Butner, J. E., May, A. M., Rugo, K. F., Harris, J. A., Oakey, D. N., Rozek, D. C., and Bryan,
A. O. (2020). Nonlinear change processes and the emergence of suicidal behavior: A conceptual model based on the fluid vulnerability theory of suicide. New ideas in psychology, 57:100758.
Canetto, S. S. and Sakinofsky, I. (1998). The gender paradox in suicide. Suicide and Life—Threatening Behavior, 28(1): 1-23.
Chandler, D. J., Jensen, P., McCall, J. G., Pickering, A. E., Schwarz, L. A., and Totah, N. K. (2019). Redefining noradrenergic neuromodulation of behavior: impacts of a modular locus coeruleus architecture. Journal of Neuroscience, 39(42): 8239-8249.
Chesney, E., Goodwin, G. M., and Fazel, S. (2014). Risks of all-cause and suicide mortality in mental disorders: a meta-review. World psychiatry, 13(2):153-160.
Clark, J. E., Watson, S., and Friston, K. J. (2018). What is mood? a computational perspective. Psychological Medicine, 48(14):2277-2284.
Clewett, D., Schoeke, A., and Mather, M. (2014). Locus coeruleus neuromodulation of memories encoded during negative or unexpected action outcomes. Neurobiology of Learning and Memory, 111:65-70.
Collins, K. A., Mendelsohn, A., Cain, C. K., and Schiller, D. (2014). Taking action in the face of threat: neural synchronization predicts adaptive coping. Journal of Neuroscience, 34(44): 14733-14738.
Constant, A., Hesp, C., Davey, C. G., Friston, K. J., and Badcock, P. B. (2021). Why depressed mood is adaptive: A numerical proof of principle for an evolutionary systems theory of depression. Computational psychiatry (Cambridge, Mass.), 5(1):60.
Cook, J. L., Swart, J. C., Froböse, M. I., Diaconescu, A. O., Geurts, D. E., Den Ouden, H. E., and Cools,
R. (2019). Catecholaminergic modulation of meta-learning. Elife, 8:e51439.
Csifcsák, G., Melsæter, E., and Mittner, M. (2020). Intermittent absence of control during reinforcement learning interferes with pavlovian bias in action selection. Journal of Cognitive Neuroscience, 32(4):646-663.
Da Costa, L., Parr, T., Sajid, N., Veselic, S., Neacsu, V., and Friston, K. (2020). Active inference on discrete state-spaces: a synthesis. Journal of Mathematical Psychology, 99:102447.
Dayan, P., Niv, Y., Seymour, B., and Daw, N. D. (2006). The misbehavior of value and the discipline of the will. Neural networks, 19(8):1153-1160.
Ding, Y., Lawrence, N., Olié, E., Cyprien, F., Le Bars, E., Bonafe, A., Phillips, M., Courtet, P., and Jollant,
F. (2015). Prefrontal cortex markers of suicidal vulnerability in mood disorders: a model-based structural neuroimaging study with a translational perspective. Translational psychiatry, 5(2):e516-e516.
Dolsen, M. R., Cheng, P., Arnedt, J. T., Swanson, L., Casement, M. D., Kim, H. S., Goldschmied, J. R., Hoffmann, R. F., Armitage, R., and Deldin, P. J. (2017).
Neurophysiological correlates of suicidal ideation in major depressive disorder: hyperarousal during sleep. Journal of affective disorders, 212:160-166.
Dolzani, S., Baratta, M., Moss, J., Leslie, N., Tilden, S., Sørensen, A., Watkins, L., Lin, Y., and Maier, S. (2018). Inhibition of a descending prefrontal circuit prevents ketamine-induced stress resilience in females. Eneuro, 5(1).
Dombrovski, A. Y., Clark, L., Siegle, G. J., Butters, M. A., Ichikawa, N., Sahakian, B. J., and Szanto, K. (2010). Reward/punishment reversal learning in older suicide attempters. American Journal of Psychiatry, 167(6):699-707.
Dombrovski, A. Y. and Hallquist, M. N. (2017). The decision neuroscience perspective on suicidal behavior: evidence and hypotheses. Current opinion in psychiatry, 30(1):7.
Dombrovski, A. Y. and Hallquist, M. N. (2021). Search for solutions, learning, simulation, and choice processes in suicidal behavior. Wiley Interdisciplinary Reviews: Cognitive Science, page e1561.
Dombrovski, A. Y., Hallquist, M. N., Brown, V. M., Wilson, J., and Szanto, K. (2019). Value-based choice, contingency learning, and suicidal behavior in mid- and late-life depression. Biological psychiatry, 85(6):506-516.
Dorfman, H. M. and Gershman, S. J. (2019). Controllability governs the balance between pavlovian and instrumental action selection. Nature communications, 10(1):1-8.
Eshel, N. and Roiser, J. P. (2010). Reward and punishment processing in depression. Biological psychiatry, 68(2):118-124.
Fallon, I. P., Tanner, M. K., Greenwood, B. N., and Baratta, M. V. (2020). Sex differences in resilience: Experiential factors and their mechanisms. European Journal of Neuroscience, 52(1):2530-2547.
FitzGerald, T. H., Dolan, R. J., and Friston, K. J. (2014). Model averaging, optimal inference, and habit formation. Frontiers in human neuroscience, 8:457.
Franklin, J. C., Ribeiro, J. D., Fox, K. R., Bentley, K. H., Kleiman, E. M., Huang, X., Musacchio, K. M., Jaroszewski, A. C., Chang, B. P., and Nock, M. K. (2017). Risk factors for suicidal thoughts and behaviors: a meta-analysis of 50 years of research. Psychological bulletin, 143(2):187.
Freeman, A., Mergl, R., Kohls, E., Székely, A., Gusmao, R., Arensman, E., Koburger, N., Hegerl, U., and Rummel-Kluge, C. (2017). A cross-national study on gender differences in suicide intent. BMC psychiatry, 17(1):1-11.
Friston, K., FitzGerald, T., Rigoli, F., Schwartenbeck, P., Pezzulo, G., et al. (2016). Active inference and learning. Neuroscience & Biobehavioral Reviews, 68:862-879.
Friston, K., Schwartenbeck, P., FitzGerald, T., Moutoussis, M., Behrens, T., and Dolan, R. J. (2013). The anatomy of choice: active inference and agency. Frontiers in human neuroscience, 7:598.
Gläscher, J., Daw, N., Dayan, P., and O'Doherty, J. P. (2010). States versus rewards: dissociable neural prediction error signals underlying model-based and model-free reinforcement learning. Neuron, 66(4):585-595.
Grahn, R. E., Hammack, S., Will, M., O'Connor, K., Deak, T., Sparks, P., Watkins, L., and Maier, S. (2002). Blockade of alpha1 adrenoreceptors in the dorsal raphe nucleus prevents enhanced conditioned fear and impaired escape performance following uncontrollable stressor exposure in rats. Behavioural brain research, 134(1-2):387-392.
Grueschow, M., Kleim, B., and Ruff, C. C. (2020). Role of the locus coeruleus arousal system in cognitive control. Journal of Neuroendocrinology, page e12890.
Harfmann, E. J., Rhyner, K. T., and Ingram, R. E. (2019). Cognitive inhibition and attentional biases in the affective go/no-go performance of depressed, suicidal populations. Journal of affective disorders, 256:228-233.
Hebb, D. O. (1955). Drives and the cns (conceptual nervous system). Psychological review, 62(4):243.
Hiser, J. and Koenigs, M. (2018). The multifaceted role of the ventromedial prefrontal cortex in emotion, decision making, social cognition, and psychopathology. Biological psychiatry, 83(8):638-647.
Holroyd, C. B. and Yeung, N. (2012). Motivation of extended behaviors by anterior cingulate cortex. Trends in cognitive sciences, 16(2): 122-128.
Hrdina, P. D., Demeter, E., Vu, T. B., Sótónyi, P., and Palkovits, M. (1993). 5-ht uptake sites and 5-ht2 receptors in brain of antidepressant-free suicide victims/depressives: increase in 5-ht2 sites in cortex and amygdala. Brain research, 614(1-2):37-44.
Huys, Q. J., Browning, M., Paulus, M. P., and Frank, M. J. (2021). Advances in the computational under-standing of mental illness. Neuropsychopharmacology, 46(1):3-19.
Isometsä, E. (2014). Suicidal behaviour in mood disorders-who, when, and why? The Canadian Journal of Psychiatry, 59(3):120-130.
Iyengar, U., Snowden, N., Asarnow, J. R., Moran, P., Tranah, T., and Ougrin, D. (2018). A further look at therapeutic interventions for suicide attempts and self-harm in adolescents: an updated systematic review of randomized controlled trials. Frontiers in psychiatry, 9:583.
Jacobs, H. I., Priovoulos, N., Poser, B. A., Pagen, L. H., Ivanov, D., Verhey, F. R., and Uludağ, K. (2020). Dynamic behavior of the locus coeruleus during arousal-related memory processing in a multi-modal 7t fmri paradigm. Elife, 9:e52059.
Jepma, M., Murphy, P. R., Nassar, M. R., Rangel-Gomez, M., Meeter, M., and Nieuwenhuis, S. (2016). Catecholaminergic regulation of learning rate in a dynamic environment. PLoS Computational Biology, 12(10):e1005171.
Joffily, M. and Coricelli, G. (2013). Emotional valence and the free-energy principle. PLoS Comput Biol, 9(6):e1003094.
Jolly, T., Trivedi, C., Adnan, M., Mansuri, Z., and Agarwal, V. (2021). Gambling in patients with major depressive disorder is associated with an elevated risk of suicide: Insights from 12-years of nationwide inpatient sample data. Addictive behaviors, 118:106872.
Jylhä, P., Rosenström, T., Mantere, O., Suominen, K., Melartin, T., Vuorilehto, M., Holma, M., Riihim″aki, K., Oquendo, M. A., Keltikangas-Järvinen, L., et al. (2016). Personality disorders and suicide attempts in unipolar and bipolar mood disorders. Journal of affective disorders, 190:632-639.
Kang, S. G., Na, K. S., Choi, J. W., Kim, J. H., Son, Y. D., and Lee, Y. J. (2017). Resting-state functional connectivity of the amygdala in suicide attempters with major depressive disorder. Progress in neuro-psychopharmacology and biological psychiatry, 77:222-227.
Karlsson, A. and Håkansson, A. (2018). Gambling disorder, increased mortality, suicidality, and associated comorbidity: A longitudinal nationwide register study. Journal of behavioral addictions, 7(4): 1091-1099.
Kerr, D. L., McLaren, D. G., Mathy, R. M., and Nitschke, J. B. (2012). Controllability modulates the anticipatory response in the human ventromedial prefrontal cortex. Frontiers in Psychology, 3:557.
Kessler, R. C., Bossarte, R. M., Luedtke, A., Zaslavsky, A. M., and Zubizarreta, J. R. (2020). Suicide pre-diction models: a critical review of recent research with recommendations for the way forward. Molecular psychiatry, 25(1): 168-179.
Klonsky, E. D. and May, A. M. (2015). The three-step theory (3st): A new theory of suicide rooted in the “ideation-to-action” framework. International Journal of Cognitive Therapy, 8(2):114-129.
Klonsky, E. D., Saffer, B. Y., and Bryan, C. J. (2018). Ideation-to-action theories of suicide: a conceptual and empirical update. Current opinion in psychology, 22:38-43.
Köhler, S., Bär, K. J., and Wagner, G. (2016). Differential involvement of brainstem noradrenergic and midbrain dopaminergic nuclei in cognitive control. Human Brain Mapping, 37(6):2305-2318.
Large, M., Kaneson, M., Myles, N., Myles, H., Gunaratne, P., and Ryan, C. (2016). Meta-analysis of longitudinal cohort studies of suicide risk assessment among psychiatric patients: heterogeneity in results and lack of improvement over time. PloS one, 11(6):e0156322.
Lawson, R. P., Bisby, J., Nord, C. L., Burgess, N., and Rees, G. (2020). The computational, pharmacological, and physiological determinants of sensory learning under uncertainty. Current Biology.
Lengvenyte, A., Oli'e, E., and Courtet, P. (2019). Suicide has many faces, so does ketamine: a narrative review on ketamine's antisuicidal actions. Current psychiatry reports, 21(12):1-10.
Linson, A. and Friston, K. (2019). Reframing ptsd for computational psychiatry with the active inference framework. Cognitive neuropsychiatry, 24(5):347-368.
Linson, A., Parr, T., and Friston, K. J. (2020). Active inference, stressors, and psychological trauma: A neuroethological model of (mal) adaptive explore-exploit dynamics in ecological context. Behavioural brain research, 380:112421.
Liu, R. T., Cheek, S. M., and Nestor, B. A. (2016). Non-suicidal self-injury and life stress: A systematic meta-analysis and theoretical elaboration. Clinical psychology review, 47:1-14.
Liu, R. T., Kleiman, E. M., Nestor, B. A., and Cheek, S. M. (2015). The hopelessness theory of depression: A quarter-century in review. Clinical Psychology: Science and Practice, 22(4):345.
Llamosas, N., Perez-Caballero, L., Berrocoso, E., Bruzos-Cidon, C., Ugedo, L., and Torrecilla, M. (2019). Ketamine promotes rapid and transient activation of ampa receptor-mediated synaptic transmission in the dorsal raphe nucleus. Progress in Neuro-Psychopharmacology and Biological Psychiatry, 88:243-252.
López-Gil, X., Jiménez-Sánnchez, L., Campa, L., Castro, E., Frago, C., and Adell, A. (2019). Role of serotonin and noradrenaline in the rapid antidepressant action of ketamine. ACS chemical neuroscience, 10(7):3318-3326.
Maier, S. F. and Seligman, M. E. (2016). Learned helplessness at fifty: Insights from neuroscience. Psycho-logical review, 123(4):349.
Mann, J. J., Currier, D., Stanley, B., Oquendo, M. A., Amsel, L. V., and Ellis, S. P. (2006). Can biological tests assist prediction of suicide in mood disorders? International Journal of Neuropsychopharmacology, 9(4):465-474.
Mann, J. J., Huang, Y. y., Underwood, M. D., Kassir, S. A., Oppenheim, S., Kelly, T. M., Dwork, A. J., and Arango, V. (2000). A serotonin transporter gene promoter polymorphism (5-httlpr) and prefrontal cortical binding in major depression and suicide. Archives of general psychiatry, 57(8):729-738.
Mann, J. J. and Rizk, M. M. (2020). A brain-centric model of suicidal behavior. American journal of psychiatry, 177(10):902-916.
Marr, D. and Poggio, T. (1976). From understanding computation to understanding neural circuitry.
Mathews, A. and MacLeod, C. (2005). Cognitive vulnerability to emotional disorders. Annu. Rev. Clin. Psychol., 1:167-195.
Matsumoto, M. and Hikosaka, O. (2009). Representation of negative motivational value in the primate lateral habenula. Nature neuroscience, 12(1):77.
Matsumoto, M., Matsumoto, K., Abe, H., and Tanaka, K. (2007). Medial prefrontal cell activity signaling prediction errors of action values. Nature neuroscience, 10(5):647-656.
May, A. M., Pachkowski, M. C., and Klonsky, E. D. (2020). Motivations for suicide: Converging evidence from clinical and community samples. Journal of psychiatric research, 123:171-177.
Meehl, P. E. (1990). Appraising and amending theories: The strategy of lakatosian defense and two principles that warrant it. Psychological inquiry, 1(2):108-141.
Mérelle, S., Foppen, E., Gilissen, R., Mokkenstorm, J., Cluitmans, R., and Van Ballegooijen, W. (2018). Characteristics associated with non-disclosure of suicidal ideation in adults. International journal of environmental research and public health, 15(5):943.
Messina, I., Sambin, M., Beschoner, P., and Viviani, R. (2016). Changing views of emotion regulation and neurobiological models of the mechanism of action of psychotherapy. Cognitive, Affective, & Behavioral Neuroscience, 16(4):571-587.
Metzger, M., Bueno, D., and Lima, L. B. (2017). The lateral habenula and the serotonergic system. Pharmacology Biochemistry and Behavior, 162:22-28.
Miller, A. B., Mclaughlin, K. A., Busso, D. S., Brueck, S., Peverill, M., and Sheridan, M. A. (2018). Neural correlates of emotion regulation and adolescent suicidal ideation. Biological psychiatry: cognitive neuroscience and neuroimaging, 3(2):125-132.
Millner, A. J., den Ouden, H. E., Gershman, S. J., Glenn, C. R., Kearns, J. C., Bornstein, A. M., Marx, B. P., Keane, T. M., and Nock, M. K. (2019). Suicidal thoughts and behaviors are associated with an increased decision-making bias for active responses to escape aversive states. Journal of abnormal psychology, 128(2):106.
Millner, A. J., Gershman, S. J., Nock, M. K., and den Ouden, H. E. (2018). Pavlovian control of escape and avoidance. Journal of Cognitive Neuroscience, 30(10):1379-1390.
Millner, A. J., Robinaugh, D. J., and Nock, M. K. (2020). Advancing the understanding of suicide: the need for formal theory and rigorous descriptive research. Trends in cognitive sciences.
Monkul, E., Hatch, J. P., Nicoletti, M. A., Spence, S., Brambilla, P., Lacerda, A. L. T. d., Sassi, R. B., Mallinger, A., Keshavan, M., and Soares, J. C. (2007). Fronto-limbic brain structures in suicidal and non-suicidal female patients with major depressive disorder. Molecular psychiatry, 12(4):360-366.
Morabito, D. M., Boffa, J. W., Bedford, C. E., Chen, J. P., and Schmidt, N. B. (2020). Hyperarousal symptoms and perceived burdensomeness interact to predict suicidal ideation among trauma-exposed individuals. Journal of psychiatric research, 130:218-223.
Naghavi, M., Abajobir, A. A., Abbafati, C., Abbas, K. M., Abd-Allah, F., Abera, S. F., Aboyans, V., Adetokunboh, O., Afshin, A., Agrawal, A., et al. (2017). Global, regional, and national age-sex specific mortality for 264 causes of death, 1980-2016: a systematic analysis for the global burden of disease study 2016. The Lancet, 390(10100): 1151-1210.
Nair, A., Rutledge, R. B., and Mason, L. (2020). Under the hood: using computational psychiatry to make psychological therapies more mechanism-focused. Frontiers in psychiatry, 11:140.
Nock, M. K., Hwang, I., Sampson, N. A., and Kessler, R. C. (2010). Mental disorders, comorbidity and suicidal behavior: results from the national comorbidity survey replication. Molecular psychiatry, 15(8):868-876.
O'Connor, R. C. and Kirtley, O. J. (2018). The integrated motivational-volitional model of suicidal behaviour. Philosophical Transactions of the Royal Society B: Biological Sciences, 373(1754):20170268.
Oli'e, E., Ding, Y., Le Bars, E., de Champfleur, N. M., Mura, T., Bonafé, A., Courtet, P., and Jollant, F. (2015). Processing of decision-making and social threat in patients with history of suicidal attempt: A neuroimaging replication study. Psychiatry Research: Neuroimaging, 234(3):369-377.
Oquendo, M. A., Bongiovi-Garcia, M. E., Galfalvy, H., Goldberg, P. H., Grunebaum, M. F., Burke, A. K., and Mann, J. J. (2007). Sex differences in clinical predictors of suicidal acts after major depression: a prospective study. American Journal of Psychiatry, 164(1):134-141.
Oquendo, M. A., Galfalvy, H. C., Choo, T. H., Kandlur, R., Burke, A. K., Sublette, M. E., Miller, J. M., Mann, J. J., and Stanley, B. H. (2020). Highly variable suicidal ideation: a phenotypic marker for stress induced suicide risk. Molecular psychiatry, pages 1-8.
Oquendo, M. A., Sullivan, G. M., Sudol, K., Baca-Garcia, E., Stanley, B. H., Sublette, M. E., and Mann, J. J. (2014). Toward a biosignature for suicide. American Journal of Psychiatry, 171(12):1259-1277.
Ougrin, D., Tranah, T., Stahl, D., Moran, P., and Asarnow, J. R. (2015). Therapeutic interventions for suicide attempts and self-harm in adolescents: systematic review and meta-analysis. Journal of the American Academy of Child & Adolescent Psychiatry, 54(2):97-107.
Pallayova, M., Brandeburova, A., and Tokarova, D. (2019). Update on sexual dimorphism in brain structure-function interrelationships: A literature review. Applied psychophysiology and biofeedback, 44(4):271-284.
Pezzulo, G., Rigoli, F., and Friston, K. (2015). Active inference, homeostatic regulation and adaptive behavioural control. Progress in neurobiology, 134:17-35.
Pham, T., Mendez-David, I., Defaix, C., Guiard, B., Tritschler, L., David, D., and Gardier, A. (2017). Ketamine treatment involves medial prefrontal cortex serotonin to induce a rapid antidepressant-like activity in balb/cj mice. Neuropharmacology, 112:198-209.
Poe, G. R., Foote, S., Eschenko, O., Johansen, J. P., Bouret, S., Aston-Jones, G., Harley, C. W., Manahan-Vaughan, D., Weinshenker, D., Valentino, R., et al. (2020). Locus coeruleus: a new look at the blue spot. Nature Reviews Neuroscience, pages 1-16.
Pudovkina, O. L., Cremers, T. I., and Westerink, B. H. (2003). Regulation of the release of serotonin in the dorsal raphe nucleus by α1 and α2 adrenoceptors. Synapse, 50(1):77-82.
Pulcu, E. and Browning, M. (2017). Affective bias as a rational response to the statistics of rewards and punishments. Elife, 6:e27879.
Pulcu, E. and Browning, M. (2019). The misestimation of uncertainty in affective disorders. Trends in Cognitive Sciences, 23(10):865-875.
Richard-Devantoy, S., Berlim, M., and Jollant, F. (2014). A meta-analysis of neuropsychological markers of vulnerability to suicidal behavior in mood disorders. Psychological medicine, 44(8): 1663-1673.
Riggs, L. M. and Gould, T. D. (2021). Ketamine and the future of rapid-acting antidepressants. Annual Review of Clinical Psychology, 17.
Rizk, M. M., Choo, T. H., Galfalvy, H., Biggs, E., Brodsky, B. S., Oquendo, M. A., Mann, J. J., and Stanley,
B. (2019). Variability in suicidal ideation is associated with affective instability in suicide attempters with borderline personality disorder. Psychiatry, 82(2):173-178.
Rushworth, M. F. and Behrens, T. E. (2008). Choice, uncertainty and value in prefrontal and cingulate cortex. Nature neuroscience, 11(4):389-397.
Sales, A. C., Friston, K. J., Jones, M. W., Pickering, A. E., and Moran, R. J. (2019). Locus coeruleus tracking of prediction errors optimises cognitive flexibility: An active inference model. PLoS computational biology, 15(1):e1006267.
Schmaal, L., van Harmelen, A. L., Chatzi, V., Lippard, E. T., Toenders, Y. J., Averill, L. A., Mazure, C. M., and Blumberg, H. P. (2020). Imaging suicidal thoughts and behaviors: a comprehensive review of 2 decades of neuroimaging studies. Molecular psychiatry, 25(2):408-427.
Sharot, T. and Garrett, N. (2016). Forming beliefs: Why valence matters. Trends in cognitive sciences, 20(1):25-33.
Shenhav, A., Botvinick, M. M., and Cohen, J. D. (2013). The expected value of control: an integrative theory of anterior cingulate cortex function. Neuron, 79(2):217-240.
Shepard, R. D., Langlois, L. D., Browne, C. A., Berenji, A., Lucki, I., and Nugent, F. S. (2018). Ketamine reverses lateral habenula neuronal dysfunction and behavioral immobility in the forced swim test following maternal deprivation in late adolescent rats. Frontiers in synaptic neuroscience, 10:39.
Silvetti, M., Vassena, E., Abrahamse, E., and Verguts, T. (2018). Dorsal anterior cingulate-brainstem ensemble as a reinforcement meta-learner. PLoS computational biology, 14(8):e1006370.
Smith, R., Badcock, P., and Friston, K. J. (2021). Recent advances in the application of predictive coding and active inference models within clinical neuroscience. Psychiatry and Clinical Neurosciences, 75(1):3-13.
Spoletini, I., Piras, F., Fagioli, S., Rubino, I. A., Martinotti, G., Siracusano, A., Caltagirone, C., and Spalletta, G. (2011). Suicidal attempts and increased right amygdala volume in schizophrenia. Schizophrenia research, 125(1):30-40.
Sterpenich, V., D'Argembeau, A., Desseilles, M., Balteau, E., Albouy, G., Vandewalle, G., Degueldre, C., Luxen, A., Collette, F., and Maquet, P. (2006). The locus ceruleus is involved in the successful retrieval of emotional memories in humans. Journal of Neuroscience, 26(28):7416-7423.
Steyn, R., Vawda, N., Wyatt, G. E., Williams, J., and Madu, S. (2013). Posttraumatic stress disorder diagnostic criteria and suicidal ideation in a south african police sample. African journal of psychiatry, 16(1): 19-22.
Stolz, D. S., Müller-Pinzler, L., Krach, S., and Paulus, F. M. (2020). Internal control beliefs shape positive affect and associated neural dynamics during outcome valuation. Nature communications, 11(1):1-13.
Szanto, K., Clark, L., Hallquist, M., Vanyukov, P., Crockett, M., and Dombrovski, A. Y. (2014). The cost of social punishment and high-lethality suicide attempts in the second half of life. Psychology and aging, 29(1):84.
Tervo, D. G., Proskurin, M., Manakov, M., Kabra, M., Vollmer, A., Branson, K., and Karpova, A. Y. (2014). Behavioral variability through stochastic choice and its gating by anterior cingulate cortex. Cell, 159(1):21-32.
Uematsu, A., Tan, B. Z., Ycu, E. A., Cuevas, J. S., Koivumaa, J., Junyent, F., Kremer, E. J., Witten, I. B., Deisseroth, K., and Johansen, J. P. (2017). Modular organization of the brainstem noradrenaline system coordinates opposing learning states. Nature neuroscience, 20(11):1602.
van Heeringen, K. and Mann, J. J. (2014). The neurobiology of suicide. The Lancet Psychiatry, 1(1):63-72.
Van Orden, K. A., Witte, T. K., Cukrowicz, K. C., Braithwaite, S. R., Selby, E. A., and Joiner Jr, T. E. (2010). The interpersonal theory of suicide. Psychological review, 117(2):575.
Vanyukov, P. M., Szanto, K., Hallquist, M. N., Siegle, G. J., Reynolds III, C. F., Forman, S. D., Aizenstein,
H. J., and Dombrovski, A. Y. (2016). Paralimbic and lateral prefrontal encoding of reward value during intertemporal choice in attempted suicide. Psychological medicine, 46(2):381.
Vassena, E., Krebs, R. M., Silvetti, M., Fias, W., and Verguts, T. (2014). Dissociating contributions of acc and vmpfc in reward prediction, outcome, and choice. Neuropsychologia, 59:112-123.
Verrocchio, M. C., Carrozzino, D., Marchetti, D., Andreasson, K., Fulcheri, M., and Bech, P. (2016). Mental pain and suicide: a systematic review of the literature. Frontiers in Psychiatry, 7:108.
Wagner, G., Koch, K., Schachtzabel, C., Schultz, C. C., Sauer, H., and Schlösser, R. G. (2011). Structural brain alterations in patients with major depressive disorder and high risk for suicide: evidence for a distinct neurobiological entity? Neuroimage, 54(2): 1607-1614.
Wang, K. S. and Delgado, M. R. (2019). Corticostriatal circuits encode the subjective value of perceived control. Cerebral Cortex, 29(12):5049-5060.
Wang, L., Zhao, Y., Edmiston, E. K., Womer, F. Y., Zhang, R., Zhao, P., Jiang, X., Wu, F., Kong, L., Zhou, Y., et al. (2020). Structural and functional abnormities of amygdala and prefrontal cortex in major depressive disorder with suicide attempts. Frontiers in psychiatry, 10:923.
Wanke, N. and Schwabe, L. (2020). Dissociable neural signatures of passive extinction and instrumental control over threatening events. Social cognitive and affective neuroscience, 15(6):625-634.
Weinberg, I., Ronningstam, E., Goldblatt, M. J., Schechter, M., Wheelis, J., and Maltsberger, J. T. (2010). Strategies in treatment of suicidality: identification of common and treatment-specific interventions in empirically supported treatment manuals. The Journal of clinical psychiatry, 71(6):699-706.
Wilkinson, S. T., Ballard, E. D., Bloch, M. H., Mathew, S. J., Murrough, J. W., Feder, A., Sos, P., Wang, G., Zarate Jr, C. A., and Sanacora, G. (2018). The effect of a single dose of intravenous ketamine on suicidal ideation: a systematic review and individual participant data meta-analysis. American journal of psychiatry, 175(2):150-158.
Willeumier, K., Taylor, D. V., and Amen, D. G. (2011). Decreased cerebral blood flow in the limbic and prefrontal cortex using spect imaging in a cohort of completed suicides. Translational psychiatry, 1(8):e28-e28.
Williams, A. V. and Trainor, B. C. (2018). The impact of sex as a biological variable in the search for novel antidepressants. Frontiers in neuroendocrinology, 50:107-117.
Yang, Y., Cui, Y., Sang, K., Dong, Y., Ni, Z., Ma, S., and Hu, H. (2018a). Ketamine blocks bursting in the lateral habenula to rapidly relieve depression. Nature, 554(7692):317-322.
Yang, Y., Wang, H., Hu, J., and Hu, H. (2018b). Lateral habenula in the pathophysiology of depression. Current opinion in neurobiology, 48:90-96.
Yerkes, R. M., Dodson, J. D., et al. (1908). The relation of strength of stimulus to rapidity of habit-formation. Punishment: Issues and experiments, pages 27-41.
Yu, A. J. and Dayan, P. (2005). Uncertainty, neuromodulation, and attention. Neuron, 46(4):681-692. Zanos, P. and Gould, T. D. (2018). Mechanisms of ketamine action as an antidepressant. Molecular psychiatry, 23(4):801-811.
Zilverstand, A., Parvaz, M. A., and Goldstein, R. Z. (2017). Neuroimaging cognitive reappraisal in clinical populations to define neural targets for enhancing emotion regulation. a systematic review. Neuroimage, 151:105-116.

What is claimed is:

1. A method for predicting active-escape bias in a mammalian subject, the method comprising:
providing a series of cues to the mammalian subject;
in association with the cues, using a physical stimulator adapted to selectively apply an aversive physical stimulus to administer to the mammalian subject, according to a predetermined pattern, a series of response states, wherein each of the response states is associated with a particular one of the cues;
responsive to each of the cues, receiving, from the mammalian subject, via a physical actuator, a physical signal, wherein the physical signal is one of:
actuation of the physical actuator; and
non-actuation of the physical actuator within a predetermined time from initiation of the cue; and
recording each received physical signal in association with the respective response state;
wherein each response state in the series of response states is selected from the group consisting of:
an active-escape state, wherein:
the aversive physical stimulus is initially applied;
the actuation of the physical actuator will, according to a first probabilistic function, do one of:
decrease the duration of the aversive physical stimulus, relative to the non-actuation of the physical actuator; and
increase the duration of the aversive physical stimulus, relative to the non-actuation of the physical actuator; and
according to the first probabilistic function, the actuation of the physical actuator is more likely to decrease the duration of the aversive physical stimulus relative to the non-actuation of the physical actuator than to increase the duration of the aversive physical stimulus relative to the non-actuation of the physical actuator;
a passive-escape state, wherein:
the aversive physical stimulus is initially applied; and
the actuation of the physical actuator will, according to a second probabilistic function, do one of:
decrease the duration of the aversive physical stimulus, relative to the non-actuation of the physical actuator; and
increase the duration of the aversive physical stimulus, relative to the non-actuation of the physical actuator; and
according to the second probabilistic function, the actuation of the physical actuator is more likely to increase the duration of the aversive physical stimulus relative to the non-actuation of the physical actuator than to decrease the duration of the aversive physical stimulus relative to the non-actuation of the physical actuator;
an active-avoid state, wherein:
the aversive physical stimulus is initially withheld;
the actuation of the physical actuator will, according to a third probabilistic function, do one of:
maintain withholding of the aversive physical stimulus; and
initiate application of the aversive physical stimulus; and
according to the third probabilistic function, the actuation of the physical actuator is more likely to maintain withholding of the aversive physical stimulus than to initiate application of the aversive physical stimulus;
a passive-avoid state, wherein:
the aversive physical stimulus is initially withheld;
the actuation of the physical actuator will, according to a fourth probabilistic function, do one of:
maintain withholding of the aversive physical stimulus; and
initiate application of the aversive physical stimulus; and
according to the fourth probabilistic function, actuation of the physical actuator is more likely to initiate application of the aversive physical stimulus than to maintain withholding of the aversive physical stimulus; and
wherein the predetermined pattern includes:
at least one first sequence in which:
the active-escape state is more likely than the passive-escape state; and
the active-avoid state is more likely than the passive-avoid state;
at least one second sequence in which:
the passive-escape state is more likely than the active-escape state; and
the passive-avoid state is more likely than the active-avoid state; and
at least one reversal between respective ones of the at least one first sequence and the at least one second sequence;
transforming the physical signals according to a predefined model that incorporates the predetermined pattern to obtain at least one learning variable of the mammalian subject;
applying the predefined model to the at least one learning variable to classify an expected cause of an individual bias of the mammalian subject toward or away from active-escape behaviour;
characterized in that:
the at least one learning variable includes at least one of:
a belief decay rate of the mammalian subject; and
a learning rate of the mammalian subject.

2. The method of claim 1, wherein the predefined model is a structured Bayesian model.

3. The method of claim 1, wherein the at least one learning variable includes a stress sensitivity parameter for the mammalian subject.

4. The method of claim 1, wherein the at least one learning variable includes a controllability threshold parameter for the mammalian subject.

5. The method of claim 1, wherein the mammalian subject is a human.

6. The method of claim 1, wherein a classification of the expected cause of the individual bias of the mammalian subject toward or away from active-escape behaviour is presented as a standardized score.

7. The method of claim 1, wherein:
during the at least one first sequence:
a likelihood of the active-escape state relative to the passive-escape state varies;
a likelihood of the active-avoid state relative to the passive-avoid state varies; and
during the at least one second sequence:
a likelihood of the passive-escape state relative to the active-escape state varies; and
a likelihood of the passive-avoid state relative to the active-avoid state varies.

8. The method of claim 1, wherein the aversive physical stimulus is selected from the group consisting of aversive aural stimulus, aversive haptic stimulus and aversive olfactory stimulus.

9. The method of claim 1, wherein the at least one reversal comprises a plurality of reversals.

10. The method of claim 1, wherein the series of cues is provided via a physical cue device including at least one of:
(a) a visual cue device comprising at least one of (i) at least one indicator light and (ii) at least one display screen;
(b) an audio cue device; or
(c) a haptic cue device.

11. The method of claim 1, wherein the physical stimulator is one of:
(a) an audio stimulator;
(b) a device that can emit an unpleasant odor; or
(c) a device that can apply an unpleasant haptic sensation.

12. The method of claim 1, wherein the physical actuator is one of a button, a lever, a joystick, a switch, a foot pedal, or a touch screen.

13. The method of claim 1, wherein the series of cues is provided by the physical stimulator.

14. An apparatus for predicting active-escape bias in a mammalian subject, the apparatus comprising:
a control device;
a physical cue device coupled to the control device;
a physical stimulator coupled to the control device and adapted to apply an aversive physical stimulus to a mammalian subject; and
a physical actuator coupled to the control device;
wherein the control device is configured to:
cause the physical cue device to provide a series of cues to the mammalian subject;
in association with the cues, cause the physical stimulator to administer to the mammalian subject, according to a predetermined pattern, a series of response states, wherein each of the response states is associated with a particular one of the cues;
responsive to each of the cues, receive, from the mammalian subject, via the physical actuator, a physical signal, wherein the physical signal is one of:
actuation of the physical actuator; and
non-actuation of the physical actuator within a predetermined time from initiation of the cue; and
record each received physical signal in association with the respective response state;
wherein each response state in the series of response states is selected from the group consisting of:
an active-escape state, wherein:
the aversive physical stimulus is initially applied;
the actuation of the physical actuator will, according to a first probabilistic function, do one of:
decrease the duration of the aversive physical stimulus, relative to the non-actuation of the physical actuator; and
increase the duration of the aversive physical stimulus, relative to the non-actuation of the physical actuator; and
according to the first probabilistic function, the actuation of the physical actuator is more likely to decrease the duration of the aversive physical stimulus relative to the non-actuation of the physical actuator than to increase the duration of the aversive physical stimulus relative to the non-actuation of the physical actuator;
a passive-escape state, wherein:
the aversive physical stimulus is initially applied; and
the actuation of the physical actuator will, according to a second probabilistic function, do one of:
decrease the duration of the aversive physical stimulus, relative to the non-actuation of the physical actuator; and
increase the duration of the aversive physical stimulus, relative to the non-actuation of the physical actuator; and
according to the second probabilistic function, the actuation of the physical actuator is more likely to increase the duration of the aversive physical stimulus relative to the non-actuation of the physical actuator than to decrease the duration of the aversive physical stimulus relative to the non-actuation of the physical actuator;
an active-avoid state, wherein:
the aversive physical stimulus is initially withheld;
the actuation of the physical actuator will, according to a third probabilistic function, do one of:
maintain withholding of the aversive physical stimulus; and
initiate application of the aversive physical stimulus; and
according to the third probabilistic function, the actuation of the physical actuator is more likely to maintain withholding of the aversive physical stimulus than to initiate application of the aversive physical stimulus;
a passive-avoid state, wherein:
the aversive physical stimulus is initially withheld;
the actuation of the physical actuator will, according to a fourth probabilistic function, do one of:
maintain withholding of the aversive physical stimulus; and
initiate application of the aversive physical stimulus; and
according to the fourth probabilistic function, actuation of the physical actuator is more likely to initiate application of the aversive physical stimulus than to maintain withholding of the aversive physical stimulus; and
wherein the predetermined pattern includes:
at least one first sequence in which:
the active-escape state is more likely than the passive-escape state; and
the active-avoid state is more likely than the passive-avoid state;
at least one second sequence in which:
the passive-escape state is more likely than the active-escape state; and
the passive-avoid state is more likely than the active-avoid state; and
at least one reversal between respective ones of the at least one first sequence and the at least one second sequence;
wherein the control device is further configured to:
transform the physical signals according to a predefined model that incorporates the predetermined pattern to obtain at least one learning variable of the mammalian subject; and
apply the predefined model to the at least one learning variable to classify an expected cause of an individual bias of the mammalian subject toward or away from active-escape behaviour;
characterized in that:
the at least one learning variable includes at least one of:
a belief decay rate of the mammalian subject; and
a learning rate of the mammalian subject.

15. The apparatus of claim 14, wherein the predefined model is a structured Bayesian model.

16. The apparatus of claim 14, wherein the at least one learning variable includes a stress sensitivity parameter for the mammalian subject.

17. The apparatus of claim 14, wherein the at least one learning variable includes a controllability threshold parameter for the mammalian subject.

18. The apparatus of claim 14, wherein the apparatus is adapted for use with a human as the mammalian subject.

19. The apparatus of claim 14, wherein a classification of the expected cause of the individual bias of the mammalian subject toward or away from active-escape behaviour is presented as a standardized score.

20. The apparatus of claim 14, wherein:
during the at least one first sequence:
a likelihood of the active-escape state relative to the passive-escape state varies;
a likelihood of the active-avoid state relative to the passive-avoid state varies; and
during the at least one second sequence:
a likelihood of the passive-escape state relative to the active-escape state varies; and
a likelihood of the passive-avoid state relative to the active-avoid state varies.

21. The apparatus of claim 14, wherein the at least one reversal comprises a plurality of reversals.

22. The apparatus of claim 14, wherein the physical cue device is one of:
(a) a visual cue device comprising at least one of (i) at least one indicator light and (ii) at least one display screen;
(b) an audio cue device; or
(c) a haptic cue device.

23. The apparatus of claim 14, wherein the physical stimulator is one of:
(a) an audio stimulator;
(b) a device that can emit an unpleasant odor; or
(c) a device that can apply an unpleasant haptic sensation.

24. The apparatus of claim 14, wherein the physical actuator is one of a button, a lever, a joystick, a switch, a foot pedal, or a touch screen.

25. The apparatus of claim 14, wherein the physical cue device and the physical stimulator comprise a single device.

26. One or more non-transitory computer-readable media comprising computer executable instructions for predicting active-escape bias in a mammalian subject, wherein the instructions which, when executed by a control device coupled to:
a physical cue device;
a physical stimulator adapted to apply an aversive physical stimulus to a mammalian subject; and
a physical actuator;
cause the control device to:
cause the physical cue device to provide a series of cues to the mammalian subject;
in association with the cues, cause the physical stimulator to administer to the mammalian subject, according to a predetermined pattern, a series of response states, wherein each of the response states is associated with a particular one of the cues;
responsive to each of the cues, receive, from the mammalian subject, via the physical actuator, a physical signal, wherein the physical signal is one of:
actuation of the physical actuator; and
non-actuation of the physical actuator within a predetermined time from initiation of the cue; and
record each received physical signal in association with the respective response state;
wherein each response state in the series of response states is selected from the group consisting of:
an active-escape state, wherein:
the aversive physical stimulus is initially applied;
the actuation of the physical actuator will, according to a first probabilistic function, do one of:
decrease the duration of the aversive physical stimulus, relative to the non-actuation of the physical actuator; and
increase the duration of the aversive physical stimulus, relative to the non-actuation of the physical actuator; and
according to the first probabilistic function, the actuation of the physical actuator is more likely to decrease the duration of the aversive physical stimulus relative to the non-actuation of the physical actuator than to increase the duration of the aversive physical stimulus relative to the non-actuation of the physical actuator;
a passive-escape state, wherein:
the aversive physical stimulus is initially applied; and
the actuation of the physical actuator will, according to a second probabilistic function, do one of:
decrease the duration of the aversive physical stimulus, relative to the non-actuation of the physical actuator; and
increase the duration of the aversive physical stimulus, relative to the non-actuation of the physical actuator; and
according to the second probabilistic function, the actuation of the physical actuator is more likely to increase the duration of the aversive physical stimulus relative to the non-actuation of the physical actuator than to decrease the duration of the aversive physical stimulus relative to the non-actuation of the physical actuator;
an active-avoid state, wherein:
the aversive physical stimulus is initially withheld;
the actuation of the physical actuator will, according to a third probabilistic function, do one of:
maintain withholding of the aversive physical stimulus; and
initiate application of the aversive physical stimulus; and
according to the third probabilistic function, the actuation of the physical actuator is more likely to maintain withholding of the aversive physical stimulus than to initiate application of the aversive physical stimulus;
a passive-avoid state, wherein:
the aversive physical stimulus is initially withheld;
the actuation of the physical actuator will, according to a fourth probabilistic function, do one of:
maintain withholding of the aversive physical stimulus; and
initiate application of the aversive physical stimulus; and
according to the fourth probabilistic function, actuation of the physical actuator is more likely to initiate application of the aversive physical stimulus than to maintain withholding of the aversive physical stimulus; and
wherein the predetermined pattern includes:
at least one first sequence in which:
the active-escape state is more likely than the passive-escape state; and
the active-avoid state is more likely than the passive-avoid state;
at least one second sequence in which:
the passive-escape state is more likely than the active-escape state; and
the passive-avoid state is more likely than the active-avoid state; and
at least one reversal between respective ones of the at least one first sequence and the at least one second sequence;
wherein the instructions further cause the control device to:
transform the physical signals according to a predefined model that incorporates the predetermined pattern to obtain at least one learning variable of the mammalian subject; and
apply the predefined model to the at least one learning variable to classify an expected cause of an individual bias of the mammalian subject toward or away from active-escape behaviour;
characterized in that:
the at least one learning variable includes at least one of:
a belief decay rate of the mammalian subject; and
a learning rate of the mammalian subject.

27. The one or more non-transitory computer-readable media of claim 26, wherein the predefined model is a structured Bayesian model.

28. The one or more non-transitory computer-readable media of claim 26, wherein the at least one learning variable includes a stress sensitivity parameter for the mammalian subject.

29. The one or more non-transitory computer-readable media of claim 26, wherein the at least one learning variable includes a controllability threshold parameter for the mammalian subject.

30. The one or more non-transitory computer-readable media of claim 26, wherein the control device is adapted for use with a human as the mammalian subject.

31. The one or more non-transitory computer-readable media of claim 26, wherein a classification of the expected cause of the individual bias of the mammalian subject toward or away from active-escape behaviour is presented as a standardized score.

32. The one or more non-transitory computer-readable media of claim 26, wherein:
during the at least one first sequence:
a likelihood of the active-escape state relative to the passive-escape state varies;
a likelihood of the active-avoid state relative to the passive-avoid state varies; and
during the at least one second sequence:
a likelihood of the passive-escape state relative to the active-escape state varies; and
a likelihood of the passive-avoid state relative to the active-avoid state varies.

33. The one or more non-transitory computer-readable media of claim 26, wherein the aversive physical stimulus is selected from the group consisting of aversive aural stimulus, aversive haptic stimulus and aversive olfactory stimulus.

34. The one or more non-transitory computer-readable media of claim 26, wherein the at least one reversal comprises a plurality of reversals.
